(19) United States
(12) Patent Application Publication
(10) Pub. No.: US 2026/0216205 A1
(43) Pub. Date: Jul. 30, 2026

(54) SYSTEMS AND METHODS FOR TREATING HEART FAILURE WITH PRESERVED EJECTION FRACTION

(71) Applicant: INVIVOSCIENCES INC., Madison, WI (US)

(72) Inventor: Tetsuro Wakatsuki, McFarland, WI (US)

(21) Appl. No.: 19/151,475

(22) PCT Filed: Jan. 31, 2024
(86) PCT No.: PCT/US2024/013686

§ 371 Date: Jul. 28, 2025

Related U.S. Application Data
(60) Provisional application No. 63/482,462, filed on Jan. 31, 2023.

(51) Int. Cl. A61K 31/551; G16H 50/20

(52) U.S. Cl. CPC A61K 31/551; G16H 50/20

(57) ABSTRACT

Disclosed herein are methods of treating selected patients of heart failure with preserved ejection fraction (HFpEF) using computer-aided patient stratification. Also disclosed herein are computer-implemented methods for classifying patients who may respond to the Rho-kinase inhibitor and show clinical efficacy.

### CROSS-REFERENCE TO RELATED APPLICATIONS

This application claims the benefit of and priority to U.S. Provisional Application No. 63/482,462, filed on Jan. 31, 2023, and the entire contents of which are incorporated herein by reference.

### STATEMENT OF GOVERNMENT INTEREST

This invention was made with government support under are R44HL139248 and R43HL164266 awarded by the National Institutes of Health. The government has certain rights in the invention.

### TECHNICAL FIELD

Described herein are systems and methods for treating heart failure in a subject having heart failure with preserved ejection fraction (HFpEF). In one aspect, described herein are methods of treating HFpEF in a subject by administering a therapeutically effective amount of a Rho-kinase (ROCK) inhibitor, an anti-fibrosis compound, or a pharmaceutically acceptable salt, ester, or solvate thereof, to the subject.

### INTRODUCTION

Heart failure (HF) is the leading cause of cardiovascular morbidity and mortality worldwide. About half of heart failure patients have heart failure with preserved ejection fraction (HFpEF). Distinct from traditional HF, i.e., heart failure with reduced ejection fraction (HFrEF) in which the ventricle cannot contract, patients with HFpEF show declined performance of heart ventricle, not at the time of contraction, but during the phase of diastole. HFpEF patients show normal ejection fraction of blood pumped out of the ventricle, but the heart muscle does not quickly relax to allow efficient filling of blood returning from the body. Morbidity and mortality of HFpEF are similar to traditional HF (HFrEF), however, therapies that benefit traditional HF are not effective in treating or preventing HP. A method for treating and/or preventing HFpEF and its complications is an essential public health goal, however, due to the complex pathophysiology of HFpEF, both the prediction and the diagnosis of this disorder remain uncertain.

### SUMMARY

In one aspect, a method of treating heart failure with preserved ejection fraction (HFpEF) is disclosed. The method of treating HFpEF may comprise administering a therapeutically effective amount of a Rho-kinase inhibitor, an anti-fibrosis compound, or a pharmaceutically acceptable salt, ester or solvate thereof, to a subject.

The Rho-kinase inhibitor may be:

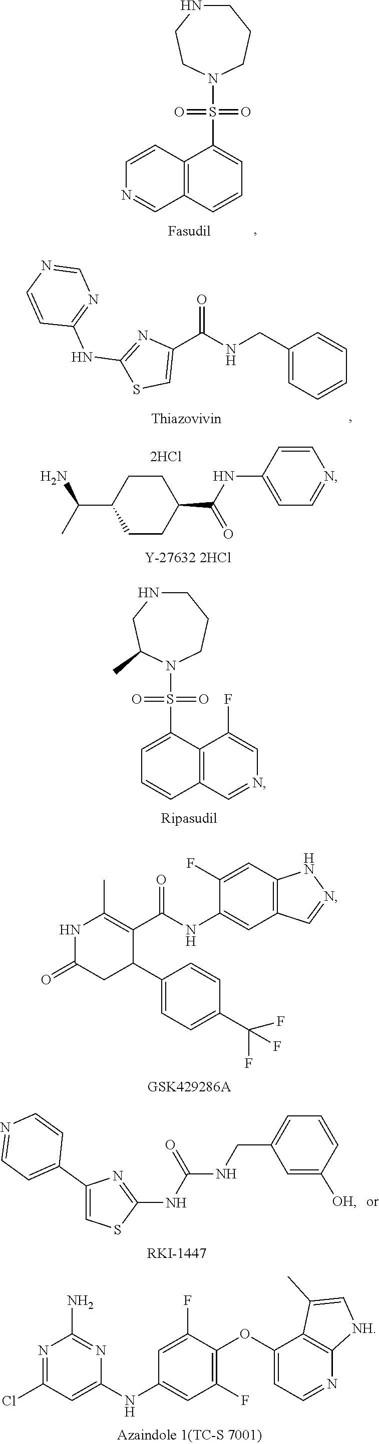

In some instances, the Rho-kinase inhibitor may be fasudil. The subject may be experiencing diastolic dysfunction, cardiac fibrosis, pulmonary hypertension, and a left ventricular ejection fraction of ≥50%. In some instances, the subject may not have cardiac amyloid deposits.

In some instances, the subject may be a human. In other instances, the subject may be an animal, such as a mammal.

In various instances, before administering the therapeutically effective amount of the Rho-kinase inhibitor, the anti-fibrosis compound, or the pharmaceutically acceptable salt, ester or solvate thereof, to the subject, a set of instructions to administer the therapeutically effective amount of the Rho-kinase inhibitor, the anti-fibrosis compound, or the pharmaceutically acceptable salt, ester or solvate thereof, to the subject is generated by a computer-implemented method.

In another aspect, a computer-implemented method is disclosed. The computer implemented method may comprise executing, by one or more processors, a feedforward artificial intelligence, the executing the machine learning algorithm comprising receiving, by one or more processors, a plurality of patient parameters, the plurality of patient parameters comprising one or more patients having diastolic dysfunction and one or more patients not having diastolic dysfunction, one or more patients having cardiac fibrosis and one or more patients not having cardiac fibrosis, one or more patients having pulmonary hypertension and one or more patients not having pulmonary hypertension, one or more patients having a left ventricular ejection fraction of ≥50% and one or more patients not having a left ventricular ejection fraction of ≥50%, and one or more patients having cardiac amyloid deposits and one or more patients not having cardiac amyloid deposits, analyzing, by one or more processors, the plurality of patient parameters, arranging, by one or more processors, the plurality of patient parameters into neurons of an input layer of an artificial neural network, applying, by one or more processors, a weight to the plurality of patient parameters arranged in the input layer, arranging, by one or more processors, the weighted plurality of patient parameters to one or more hidden layers of the feedforward artificial neural network, and generating, by one or more processors, a plurality of optimized data sets of patient parameters; executing, by one or more processors, a cluster analysis, the executing comprising: receiving, by one or more processors, a plurality of optimized data sets of patient parameters, wherein the plurality of optimized data sets of patient parameters includes data of a plurality of patients; analyzing, by one or more processors, the plurality of optimized data sets of patient parameters, identifying, by one or more processors, a plurality of populations among the plurality of optimized data sets of patient parameters, and generating, by one or more processors, one or more clusters of the plurality of populations, analyzing, by one or more processors, the one or more clusters of the plurality of populations generated by the cluster analysis, and generating, by one or more processors, a set of instructions to administer an effective amount of a Rho-kinase inhibitor, an anti-fibrosis compound, or a pharmaceutically acceptable salt, ester or solvate thereof, to a subject experiencing diastolic dysfunction, cardiac fibrosis, pulmonary hypertension, and a left ventricular ejection fraction of ≥50%, wherein the subject does not have cardiac amyloid deposits, and administering to the subject the therapeutically effective amount of the Rho-kinase inhibitor, the anti-fibrosis compound, or the pharmaceutically acceptable salt, ester or solvate thereof.

In another aspect, a method of reducing the stiffness of heart tissue is disclosed. The method of reducing the stiffness of heart tissue may comprise administering a therapeutically effective amount of a Rho-kinase inhibitor, an anti-fibrosis compound, or a pharmaceutically acceptable salt, ester, or solvate thereof, to a subject experiencing diastolic dysfunction, cardiac fibrosis, pulmonary hypertension, and a left ventricular ejection fraction of ≥50%, and wherein the subject does not have cardiac amyloid deposits.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

The patent or application file contains at least one drawing executed in color. Copies of this patent or patent application publication with color drawing(s) will be provided to the United States Patent and Trademark Office (the “Office”) upon request and payment of the necessary fee.

FIG. 1 shows the chemical structures of various exemplary Rho-kinase (ROCK) inhibitors.

FIG. 2 is a table listing various output model parameters.

FIG. 3A is a graph showing the distribution of diastolic pulmonary artery (PA) pressure in patients.

FIG. 3B is a graph showing shows the distribution of left ventricular (LV) passive stiffness in patients.

FIG. 4A is a scheme illustrating the development of personalized in vitro heart failure disease models.

FIG. 4B is a scheme illustrating the identification of heart failure patients best represented by an in vivo animal model.

FIG. 5 is a block diagram illustrating the computing environment for exemplary computer-implemented methods of the present disclosure.

FIG. 6 is a block diagram illustrating an exemplary computer system and server area network (SAN) for exemplary computer-implemented methods of the present disclosure.

FIG. 7A graphically shows length tension relationships of cardiac stress of human micro-heart tissues treated with fasudil.

FIG. 7B graphically shows length tension relationships of diastolic stiffness of human micro-heart tissues treated with fasudil.

FIG. 8A is a table of genetic data showing that the absence of ROCK1 and ROCK2 changed gene expression profiles of various genes. The absence of ROCK2 down-regulated genes that involve in fibrosis.

FIG. 8B is a western blot analysis of procollagen and alpha smooth muscle (αSM) actin expression.

FIG. 8C is a bar graph illustrating that alpha smooth muscle expression indicates a presence of myofibroblasts that are present in fibrotic tissues. Two different short hairpin RNAs (shRNAs) targeting ROCK2 (shROCK2_3 and shROCK2_5) downregulated αSM expression.

FIG. 8D is a bar graph illustrating that inhibiting ROCK2 expression down-regulates pro-collagen I protein expression.

FIG. 9A schematically illustrates automation of the cardiac cell production from induce pluripotent stem cells.

FIG. 9B shows a top view of a micro-heart tissue in a 96-well plate on day −1.

FIG. 9C shows a top view of a micro-heart tissue in a 96-well plate on day 0.

FIG. 9D is a schematic illustrating how cells or tissues growing microplates are analyzed using a robotic system.

FIG. 10A is a response curve for isoproterenol (ISO) treated matured micro-heart tissue.

FIG. 10B is a response curve for Bay K8644 treated matured micro-heart tissue.

FIG. 10C is a response curve for Milrinone treated matured micro-heart tissue.

FIG. 10D is a response curve for Dobutamine treated matured micro-heart tissue.

FIG. 11A is a confocal image of 35-day old NuHeart™ micro-heart tissue without Transforming growth factor beta (TGFβ), Red=cardiac troponin I, Green=αSM actin, Blue=4′,6-diamidino-2-phenylindole (DAPI), Double arrows: multi-nucleation (Bar=10 mm).

FIG. 11B is a confocal image of 35-day old NuHeart™ micro-heart tissue with Transforming growth factor beta (TGFβ), Red=cardiac troponin I, Green=αSM actin, Blue=DAPI, Double arrows: multi-nucleation (Bar=10 mm).

FIG. 11C-11F graphically show length-tension relationships of cardiac systolic (FIG. 10C, FIG. 10D) and diastolic (FIG. 10E, FIG. 10F) curves, monitored>30 days with and without TGFβ (10 ng/mL).

FIG. 11G is a bar graph illustrating that the positive inotropic response to ISO (0.1 mM, 1 mM) were compared with and without TGFβ (bars=s.d., n=7) at the end of 35-day tissue development.

FIG. 11H shows an elevated αSM actin in TGFβ treated sample.

FIG. 11I-11J show profiles of cardiac contractile force with and without TGFβ. ISO treatment increased the contractile amplitude and shortened the duration (chrotropic response) of NuHeart™ micro-heart tissue without TGFβ. However, the TGFβ had a blunted response to ISO.

FIG. 11I shows a profile of cardiac contractile force without TGFβ.

FIG. 11J shows a profile of cardiac contractile force with TGFβ.

FIG. 12A-12B show different perspectives of an exemplary mechanical assay device, Palpator™, InvivoSciences.

FIG. 12A shows a zoomed-in perspective of Palpator™, InvivoSciences where micro-heart tissue in a 96-well plate is analyzed.

FIG. 12B shows a zoomed-out perspective of the Palpator™, InvivoSciences instrument.

FIG. 12C shows a force probe approaching vertically stretching micro-heart tissue (top), and the plot of force (mN) responses over time (seconds) as tissue stretches (bottom).

FIG. 13A exemplifies a microplate reader used to analyze the calcium transient and action potentials of micro-heart tissues in 96-well plates.

FIG. 13B-13C show micro-heart tissues in 96-well plates stained with biological dyes (FIG. 13B) to analyze their Ca-transients and action potentials (FIG. 13C).

FIG. 13B is a photograph of an example 96-well plate containing micro-heart tissues stained with biological dyes.

FIG. 13C shows an example of the Ca-transient and action potential output for the micro-tissue analyzed.

FIG. 14A graphically shows cardiac stress of micro-heart tissue derived from human iPSCs with familial cardiac hypertrophy (FCH) and its control. Different levels of stretches were applied corresponding to the estimated sarcomere lengths within its physiological range (<2.2 μm)

FIG. 14B is a graph showing that in baseline stress of micro-heart tissue (n=6, error bar: S.D.), differences between control and FCH (familial hypertrophic cardiomyopathy (HCM)) are statistically significant (P<0.05).

FIGS. 15A-15C illustrate that a closed-loop CV model simulates a patient-specific cardiovascular (CV) function using 11 clinical parameters collected by trans-thoracic echocardiography (TTE) and right heart catheterization (RHC). The simulation defines each patient's CV phenotype with 9 model parameters. Mechanistic model parameters include E_{LV}: left ventricular (LV) contractility, l_{LV}: LV stiffness, E_{RV}: right ventricular (RV) contractility, l_{RV}: RV stiffness, E_{PA}: Pulmonary arteries (PA) stiffness, E_{PV}: Pulmonary veins (PV) stiffness, Rₚᵤₗ: Pulmonary resistance, E_{SA}: Systemic arteries (SA) stiffness, R_{sys}: Systemic resistance.

FIG. 15A is a flow chart illustrating the steps for optimization of adjustable parameters in dimensional reduction and clustering algorithm identify optimized patients cluster with distinct phenotype.

FIG. 15B shows plotted example clusters of HF patients into 19 groups including outliers (dark blue, #−1) from 136 HF patients that were clustered into similar groups without supervision using Uniform Manifold Approximation and Projection (UMAP) dimension reduction algorithm data.

FIG. 15C shows plotted ejection fraction, mean pulmonary artery pressure (mPAP), pulmonary artery wedge pressure (APWP), LV contractility, LV stiffness, and systemic arteries stiffness for each cluster.

FIGS. 16A-16D graphically illustrate the long-term effects of fasudil. The rates of stretch relative to the unstretched tissue length are depicted as strain (error bar=standard deviation of error). Number of samples are 2 or 4 in each condition.

FIG. 16A shows plotted cardiac contractility stress versus strain for twenty day-old engineered heart tissues without TGFβ that were administered with fasudil (0 μM, 1 μM, 3 μM, 10 μM: closed circle, open circle, open triangle, open square).

FIG. 16B shows plotted cardiac contractility stress versus strain for twenty day-old engineered heart tissues with TGFβ (1 ng/mL) administered with fasudil (0 μM, 1 μM, 3 μM, 10 μM: closed circle, open circle, open triangle, open square).

FIG. 16C shows plotted cardiac passive stress versus strain for twenty day-old engineered heart tissues without TGFβ that were administered with fasudil (0 M, 1 μM, 3 μM, 10 μM: closed circle, open circle, open triangle, open square).

FIG. 16D shows plotted cardiac passive stress versus strain for twenty day-old engineered heart tissues with 1 TGFβ (1 ng/mL) that were administered with fasudil (0 μM, 1 μM, 3 μM, 10 μM: closed circle, open circle, open triangle, open square).

### DETAILED DESCRIPTION

Exemplary materials, methods and techniques disclosed and contemplated herein generally relate to systems and methods for treating heart failure with preserved ejection fraction (HFpEF).

### I. DEFINITIONS

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” is used to indicate that exact values are not necessarily attainable. Therefore, the term “about” is used to indicate this uncertainty limit. The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5-1.4. The modifier “about” should also be considered as disclosing the range defined by the absolute values of the two endpoints. For example, the expression “from about 2 to about 4” also discloses the range “from 2 to 4.”

For the recitation of numeric ranges herein, each intervening number there between with the same degree of precision is contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated. For another example, when a pressure range is described as between ambient pressure and another pressure, a pressure that is ambient pressure is expressly contemplated.

As used herein, the term “subject” refers to a human or an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a human. In another embodiment, the subject is an animal.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease.

Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof.

“Suppressing” a disease or disorder involves administering a cell, composition, or compound described herein to a subject after induction of the disease or disorder thereof but before its clinical appearance or symptoms thereof have manifest.

As used herein, the terms “salt” or “salts” refers to an acid addition or base addition salt of a compound of the disclosure. “Salts” include in particular “pharmaceutical acceptable salts.” The term “pharmaceutically acceptable salts” refers to salts that retain the biological effectiveness and properties of the compounds of this disclosure and, which typically are not biologically or otherwise undesirable. In many cases, the compounds described herein may form acid and/or base salts by virtue of the presence of amino and/or carboxyl groups or groups similar thereto.

### II. HEART FAILURE WITH PRESERVED EJECTION FRACTION (HFPEF)

Heart failure is divided into two different types: heart failure due to reduced ejection fraction (HFrEF; also known as heart failure due to left ventricular systolic dysfunction or systolic heart failure) and heart failure with preserved ejection fraction (HFpEF; also known as diastolic heart failure or heart failure with normal ejection fraction). HFrEF occurs when the ejection fraction is less than 40%. Ejection fraction (EF) is a measure of pumping efficiency of a heart, or the ability of the heart to pump blood out of the left ventricle and into the circulatory system of the body. EF is commonly measured as a percentage of the blood expelled from the left ventricle during the contraction phase of a heartbeat to the total capacity of the left ventricle. An EF of a normal-functioning heart may be between 50% and 70%. Preserved EF typically refers to a measure of 50% or higher. Reduced EF typically refers to a measure of 40% or lower. An EF between 40% and 50% may be considered borderline. In other examples, reduced EF may refer to a measure below 50%, and preserved EF may refer to a measure above 50%.

Cardiac fibrosis is central to the pathology of heart failure, particularly heart failure with preserved ejection fraction (HFpEF) and is characterized by a disproportionate accumulation of fibrillated collagen that occurs after myocyte death, inflammation, enhanced workload, hypertrophy, and stimulation by a number of hormones, cytokines, and growth factors.

Cardiac fibrosis may also refer to an abnormal thickening of the heart valves due to inappropriate proliferation of cardiac fibroblasts but more commonly refers to the proliferation of fibroblasts in the cardiac muscle. Fibrocyte cells normally secrete collagen, and function to provide structural support for the heart. When over-activated this process causes thickening and fibrosis of the valve, with white tissue building up primarily on the tricuspid valve, but also occurring on the pulmonary valve. The thickening and loss of flexibility eventually may lead to valvular dysfunction and right-sided heart failure.

Elevated N-terminal pro-B-type natriuretic peptide (NT-proBNP) levels may also be associated with HFpEF. NT-proBNP is a type of biomarker often used in clinical trials to assess the severity of heart failure. Typically, a subject experiencing elevated NT-proBNP levels has NT-proBNP at >900 ng/L. In some instances, a subject experiencing elevated NT-proBNP levels has NT-proBNP at >2,000 ng/L or at >3,000 ng/L in the plasma.

The disclosed compounds and compositions may be used in methods for treatment of heart failure with preserved ejection fraction (HFpEF). The methods of treatment may comprise administering to a subject in need of such treatment a composition comprising a therapeutically effective amount of a Rho-kinase (ROCK) inhibitor, an anti-fibrosis compound, or a pharmaceutically acceptable salt, ester or solvate thereof, to the subject.

The compositions may be administered to a subject in need thereof to modulate ROCK, for a variety of diverse biological processes. The present disclosure is directed to methods for administering the composition to inhibit ROCK, an enzyme that plays a role in regulating downstream signal transduction pathways and biological activities, including smooth muscle cells' contractility.

### III. RHO-KINASE (ROCK) INHIBITORS AND OTHER ANTI-FIBROSIS COMPOUNDS

Exemplary methods of treating heart failure with preserved ejection fraction (HFpEF) in a subject may comprise administering a therapeutically effective amount of a Rho-kinase (ROCK) inhibitor, an anti-fibrosis compound, or a pharmaceutically acceptable salt, ester or solvate thereof, to the subject. The subject may be experiencing diastolic dysfunction, cardiac fibrosis, pulmonary hypertension, a left ventricular ejection fraction of >50%, and elevated N-terminal pro-B-type natriuretic peptide (NT-proBNP) levels. In various instances, the subject may not have cardiac amyloid deposits, i.e., the subject may be “amyloid negative.”

### A. Rho-Kinase (ROCK) Inhibitors

As used herein the term “Rho-kinase (ROCK) inhibitor” is a protein, nucleic acid, small molecule, antibody, or other agent that prevents expression of ROCK or down-regulates ROCK activity, such as its kinase activity. Examples of ROCK inhibitors are disclosed herein.

ROCK kinase inhibitors, such as fasudil, as used herein, may mediate myocardium (heart muscle) contractility, and reduce myocardial tissue stiffness associated with heart failure.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result may be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein the term “a therapeutically effective amount” of a Rho-kinase (ROCK) inhibitor described herein refers to an amount of the inhibitor described herein that will elicit the biological or medical response of a subject, for example, reduction or inhibition, or increase or stimulation of an enzyme or a protein activity, or ameliorate symptoms, alleviate conditions, slow or delay disease progression, or prevent a disease, etc. In one non-limiting embodiment, the term “a therapeutically effective amount” refers to the amount of the ROCK inhibitor (e.g., fasudil) described herein that, when administered to a subject, is effective to inhibit expression and/or activity of Rho-kinase (ROCK). For example, a therapeutically effective amount of a ROCK inhibitor (e.g., fasudil), may be a daily dosage of 10 mg/kg to 60 mg/kg of body weight.

In various instances, the ROCK inhibitor administered may be fasudil. In various instances, fasudil may be orally administered. In some instances, fasudil may be administered daily at a dosage of 30 mg/kg to 60 mg/kg, 35 mg/kg to 55 mg/kg or 40 mg/kg to 50 mg/kg. In some instances, fasudil may be administered daily at a dosage of no greater than 60 mg/kg, no greater than 55 mg/kg, no greater than 50 mg/kg, no greater than 45 mg/kg, no greater than 40 mg/kg, no greater than 35 mg/kg, or no greater than 30 mg/kg. In some instances, fasudil may be administered daily at a dosage of no less than 30 mg/kg, no less than 35 mg/kg, no less than 40 mg/kg, no less than 45 mg/kg, no less than 50 mg/kg, no less than 55 mg/kg, or no less than 60 mg/kg.

### B. Other Anti-Fibrosis Compounds

Experimental screening for other anti-fibrosis compounds recognized three Angiotensin II receptor blockers, two A.C.E. inhibitors, two statins, one beta-blocker, and two NSAIDs as positive hits. In addition, the screening identified various new classes of compounds with different targets and mechanisms of action for the potential treatment for cardiac fibrosis. Many of the compounds modulate neurotransmitter receptors, such as 1) histamine receptors, 2) serotonin receptors, 3) dopamine receptors, 4) glutamine receptors, and 5) GABA receptors.

Other classes of anti-fibrosis compounds may modulate targets, such as 1) Glucose metabolism, 2) adrenergic receptors, 3) Retinoic acid, 4) Ion channels, 5) K_{ATP} channel, 6) Prostaglandin, 7) Phosphodiesterase, 8) Bisphosphonate, 9) Anti-viral, 9) DNA function, 10) Sex hormone. 11) HIV-protease, 12) Antibiotics, 13) EGF-receptor, 14) Thrombin function, 15) Rho kinase, 16) Cholecystokinin, 17) other targets. Exemplary compounds that may be used to modulate the targets include the following:
005 Losartan Angiotensin receptor blocker (AT1)
007 Telmisartan Angiotensin receptor blocker (AT1)
057 Olmesartan Angiotensin receptor blocker (AT1)
054 Lisinopril Angiotensin-converting enzyme (A.C.E.) inhibitor
082 Ramipril Angiotensin-converting enzyme (A.C.E.) inhibitor
025 Simvastatin HMG-CoA reductase Inhibitor
091 Pravastatin HMG-CoA reductase inhibitor
041 Propranolol beta-adrenergic receptor-blocking agent
018 Acemetacin Non-steroidal anti-inflammatory drug
090 Sulindac Non-steroidal anti-inflammatory drug
001 Metformin Not clear molecular mechanism
095 Pioglitazone DPP-4 inhibitor
002 Tiotidine Histamine H2 receptor antagonist
006 Cimetidine Histamine H2 receptor antagonist
012 Roxatidine Histamine H2 receptor antagonist
066 Triprolidine A first generation histamine H1 antagonist
003 Tranilast Inhibiting the release of histamine from mast cells
Serotonin 5-HT, 5-hydroxytrptmamine Receptor Modulators
004 Memantine N-methyl-D-aspartate (NMDA) receptor antagonist
017 Zolmitriptan A novel 5HT(1B/1D) receptor agonist
037 Ergotamine Activating 5-HT(1B/D/F) receptors
038 Nefazodone Potent 5-HT(2A/2C) receptor antagonist
042 Methysergide 5HT2 antagonist, it is also a 5HT1 agonist
013 Deprenyl Inhibitor of MAO-B, blocking the breakdown of dopamine
021 Bupropion Norepinephrine-dopamine reuptake inhibitor
024 Lobeline VMAT2 ligand, which stimulates dopamine release
069 Itopride Dopamine D2 receptor antagonist
030 Apomorphine Non-ergoline dopamine agonist
087 Spiperone Selective D2 dopamine receptor antagonist
078 Quinpirole A selective D2 and D3 receptor agonist
014 Aniracetam A positive modulator of AMPA-sensitive glutamate receptors
023 Bethanechol Selective agonist of the muscarinic acetylcholine receptor
034 Carbachol Stimulates both muscarinic and nicotinic receptors
040 Tubocurarine Antagonist of nicotinic acetylcholine receptor
093 Pancuronium Competitive antagonist of nicotinic acetylcholine receptor
038 Physostigmine Reversible cholinesterase inhibitor
043 Tacrine Reversible cholinesterase inhibitor
050 Rocuronium Competitively bind to nicotinic cholinergic receptors
044 Propofol Positive modulation of GABA-A receptors
053 Ivermectin An agonist of GABA
081 Tramadol Selective, weak OP3-receptor agonists
015 Epinephrine Hormone activate adrenergic receptor, relaxing smooth muscle
072 Melatonin A hormone synthesized and released from the pineal gland
049 Niguldipine Selective antagonist for the alA-adrenoceptor
079 Xylazine A potent alpha-2 adrenergic agonist. An antihypertensive agent.
064 Fluperlapine Marked affinity for alpha-1 adrenoceptors (IC₅₀~10 nM)
076 Tulobuterol Long-acting beta2-adrenergic receptor agonist
080 Salmeterol A long-acting beta2-adrenergic agonist
008 Retinoic acid Metabolite of vitamin A1 (all-trans-retinol)
084 Vitamin A1 Retinol to treat and prevent vitamin A deficiency.
009 Dofetilide IKr blocker
068 Indapamide Antihypertensive and a diuretic
038 Nifekalant A nonselective K⁺ channel blocker
010 Lidocaine Blocking the fast voltage-gated Na⁺ channels
016 Ouabain Cardiac glycoside similar to digitoxin
022 Nimodipine L-type Ca channel inhibitor
028 Riluzole Inhibit glutamate release
030 Zonisamide Block sodium and calcium channels
059 Glimepiride Blocks KATP channel
060 Butyrylcholine Blocks KATP channel
086 Nateglinide Blocks KATP channel
011 Alprostadil Prostaglandin E1 (PGE1)
052 Sulfasalazine Mechanism is unclear. Inhibition of prostaglandins
019 Zaprinast Selective inhibitor of cGMP)-dependent phosphodiesterases,
020 Vardenafil Inhibitor of cGMP-dependent phosphodiesterase type 5 (PDE5)
032 Trequinsin Potent PDE3 inhibitor
074 Zardaverine A dual-selective inhibitor of phosphodiesterase III/IV
083 Siguazodan A selective inhibitor of phosphodiesterase 3
029 Lorglumide Inhibitor of the cholecystokinin-A and -B receptors
Bisphosphonate (known relative potency Zoledronate>Pamidronate=Alendronate)
026 Zoledronate Bisphosphonate for osteoporosis,
065 Pamidronate Bisphosphonate for osteoporosis
092 Alendronate Bisphosphonate for osteoporosis
027 Oseltamivir Inhibitor of influenza virus
046 Rimantadine M2 ion channel inhibitor
047 Allopurinol Xanthine oxidase inhibitor
048 Itraconazole Azole antifungal agent
063 Idoxuridine Antiviral agent use in keratitis caused by herpes simplex virus.
089 Ribavirin Synthetic guanosine nucleoside and antiviral agent
031 Acycloguanosine Nucleoside analog D.N.A. polymerase inhibitor
035 Shikonin Inhibitor of D.N.A.
067 Fluorouracil An antineoplastic anti-metabolite
073 Lomustine For neoplastic diseases, an alkylating agent.
033 Goserelin Gonadotropin releasing hormone agonist
062 Hexestrol Estrogen receptor agonist
085 Stanozolol Anabolic steroid
036 Imiquimod A toll-like receptor 7 agonist
039 Nelfinavir Inhibitor of HIV-1 protease
075 Amprenavir Inhibitor of HIV-1 protease
045 Streptomycin Antibiotic
056 Florfenicol Antibiotic
071 Pefloxacin Antibiotic
094 Rifamycin Antibiotic
051 Tanshinone A derivative of phenanthrene-quinone
055 Suramin Mechanism is unclear.
058 Mebendazole Inhibiting the synthesis of microtubules
077 Argatroban A synthetic direct thrombin inhibitor derived from L-arginine
088 Lapatinib A dual inhibitor of the EGFR HER2 receptor tyrosine kinases
096 Fasudil A potent Rho-kinase inhibitor

In various instances, the ROCK inhibitor may be fasudil.

### C. Pharmaceutical Salts, Esters, and Solvates

The disclosed compounds may exist as pharmaceutically acceptable salts, esters, or solvates.

As used herein, the term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts may be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

As used herein, the term “a pharmaceutically acceptable solvate” of the compound of refers to a substance formed by its association with solvent molecule(s). For example, the compounds described herein may form a hydrate with water.

### D. Pharmaceutical Compositions

The ROCK inhibitors (e.g., fasudil) and other compounds disclosed herein may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure (e.g., fasudil) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material, or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; tale; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions. Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%. Suitable colorants include a colorant such as an FD&C dye. When used, the amount of colorant in a systemic or topical composition is typically about 0.005 to about 0.1%. Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%. Suitable antioxidants include butylated hydroxyanisole (“BHA”), butylated hydroxytoluene (“BHT”), and vitamin E. The amount of antioxidant(s) in a systemic or topical composition is typically about 0.1 to about 5%. Suitable preservatives include benzalkonium chloride, methyl paraben and sodium benzoate. The amount of preservative(s) in a systemic or topical composition is typically about 0.01 to about 5%. Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%. Suitable suspending agents include AVICEL RC-591 (from FMC Corporation of Philadelphia, PA) and sodium alginate. The amount of suspending agent(s) in a systemic or topical composition is typically about 1 to about 8%. Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active (e.g., fasudil) and 50% to 99.99% of one or more carriers.

Compositions for parenteral administration typically include 0.1% to 10% of actives and 90% to 99.9% of a carrier including a diluent and a solvent.

Compositions for oral administration may have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets may be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which may be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., fasudil), and a carrier including one or more diluents disclosed above in a capsule comprising gelatin. Granules typically comprise a disclosed compound, and preferably glidants such as silicon dioxide to improve flow characteristics. Implants may be of the biodegradable or the non-biodegradable type.

The selection of ingredients in the carrier for oral compositions depends on secondary considerations like taste, cost, and shelf stability, which are not critical for the purposes of this disclosure. Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration may have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

### IV. COMPUTER-IMPLEMENTED METHODS

In various instances, a computer-implemented method is used to identify a personalized therapeutic strategy, such as a personalized therapeutic strategy to improve a heart failure patient's disease phenotype. The computer-implemented method includes the AI-Assisted, Systems-biology Integrated patient Stratification Technology (AASIST), described in more detail in the Experimental Examples section below.

Exemplary computer-implemented methods may comprise executing, by one or more processors, an artificial intelligence. Executing the artificial intelligence may comprise receiving, by one or more processors, a plurality of patient parameters comprising data. The plurality of patient parameters received by the artificial intelligence may include one or more patients having diastolic dysfunction and one or more patients not having diastolic dysfunction, one or more patients having cardiac fibrosis, and one or more patients not having cardiac fibrosis, one or more having pulmonary hypertension, and one or more patients not having pulmonary hypertension, one or more patients having a left ventricular ejection fraction of ≥50%, and one or more patients not having a left ventricular ejection fraction of ≥50%, and one or more patients having cardiac amyloid deposits and one or more patients not having cardiac amyloid deposits.

Exemplary computer-implemented methods may comprise executing, by one or more processors, a feedforward artificial neural network. Executing the feedforward artificial neural network may comprise receiving, by one or more processors, a plurality of patient parameters comprising data and metadata. The plurality of patient parameters may include one or more patients having diastolic dysfunction and one or more patients not having diastolic dysfunction, one or more patients having cardiac fibrosis, and one or more patients not having cardiac fibrosis, one or more having pulmonary hypertension, and one or more patients not having pulmonary hypertension, one or more patients having a left ventricular ejection fraction of ≥50%, and one or more patients not having a left ventricular ejection fraction of ≥50%, and artone or more patients having cardiac amyloid deposits and one or more patients not having cardiac amyloid deposits.

Exemplary computer-implemented methods may further comprise analyzing and arranging the plurality of patient parameters into neurons of an input layer of an artificial neural network. Then, a weight may be applied to the plurality of patient parameters arranged in the input layer, arranging the weighted plurality of patient parameters to one or more hidden layers of the feedforward artificial neural network, and generating a plurality of optimized data sets of patient parameters. A cluster analysis may then be executed by receiving and analyzing the plurality of optimized data sets of patient parameters. Then, by one or more processors, a plurality of populations may be identified among the plurality of optimized data sets of patient parameters, and one or more clusters of the plurality of populations may be generated and analyzed.

In various instances, the computer-implemented method includes the AI-Assisted, Systems-biology Integrated patient Stratification Technology (AASIST), described in more detail in the Experimental Examples section below.

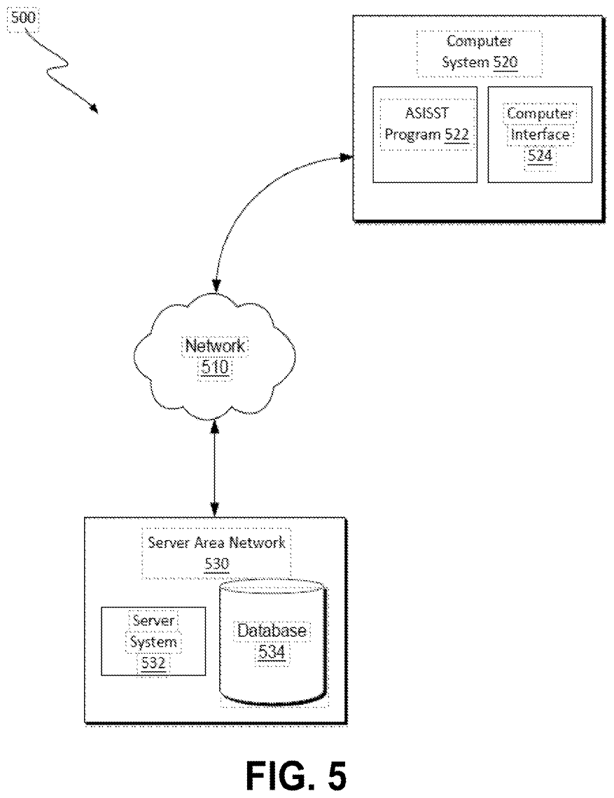

FIG. 5 is a functional block diagram schematically illustrating an exemplary computing environment 500. As shown in FIG. 5, the exemplary computing environment 500 may include a computer system 520 and a storage area network 530 connected over a network 510.

An exemplary computer system 520 may include an AASIST program 522 and a computer interface 524. The storage area network 530 includes a server system 532 and a database 534. In various instances, the computer system 520 may be a computing device that is a standalone device, a server, a laptop computer, a tablet computer, a netbook computer, a personal computer (PC), a personal digital assistant, a desktop computer, or another programmable electronic device capable of receiving, sending, and processing data. In general, the computer system 520 represents any programmable electronic device or combination of programmable electronic devices capable of executing machine readable program instructions and communications with various other computer systems (not shown). In some instances, the computer system 520 represents a computing system utilizing clustered computers and components to act as a single pool of seamless resources. The computer system 520 may be any computing device or a combination of devices with access to various other computing systems (not shown) and may execute the AASIST program 522 and the computer interface 524. For example, the computer system 520 may include various internal and external hardware components (not shown).

As depicted in FIG. 5, in some instances, the AASIST program 522 and the computer interface 124 may be stored on computer system 520. However, in other instances (not shown), the AASIST program 522 and the computer interface 524 may be stored externally and accessed through a communication network, such as network 510.

In general, network 510 may be any combination of connections and protocols that will support communications between the computer system 520, the storage area network 530, and various other computer systems (not shown), in accordance with a desired embodiment of the present disclosure. The network 510 may be, for example, a local area network (LAN), a wide area network (WAN) such as the Internet, or a combination thereof. In some instances, network 510 is a wired network, a wireless network, or a fiber optic network.

In various instances, the various other computer systems (not shown) may be a standalone device, a server, a laptop computer, a tablet computer, a netbook computer, a personal computer (PC), a desktop computer, or any programmable electronic device capable of receiving, sending, and processing data, In other instances, the various other computer systems represent a computing system utilizing clustered computers and components to act as a single pool of seamless resources. In general, the various other computer systems may be any computing device or combination of devices with access to the computer system 520 and the network 510 and may execute the AASIST program 522 and the computer interface 524. The various other computer systems may include internal and external hardware components, as depicted, and described in further detail with respect to FIG. 5.

As depicted in FIG. 5, in some instances, the AASIST program 522 utilizes, at least in part, data stored on the database 534 to manage access to the computer system 520 in response to a digital media recognition request from a user (i.e., from a user of computer system 520, alternatively referred to herein as “requestor”). More specifically, the AASIST program 522 may define one or more artifacts and weights that represent the types of input/output (I/O) that constitute a feedforward artificial neural network that generates a plurality of optimized data sets of patient parameters that are further utilized by a cluster analysis to generate a set of instructions to administer an effective amount of a Rho-kinase (ROCK) inhibitor, an anti-fibrosis compound, or a pharmaceutically acceptable salt, ester or solvate thereof, to a subject. For example, the weight may be a value analysis metric, and the artifacts may be data points associated with the one or more patient parameters.

The computer system 520 may include numerous logics and/or programs that may be managed in accordance with the AASIST program 522. In general, the computer system 520 manages access to the AASIST program 522, which represents a physical or virtual resource. In various instances, the AASIST program 522 includes certain information and code that, when executed, enables computer system 520 to take specific action with respect to another physical resource and/or virtual resource based, at least in part, on that certain information. For example, in some instances, the AASIST program 522 manages, at least in part, the ability of the computer system 520 to take various actions with respect to one or more physical resources and/or virtual resources. In some instances, the AASIST program 522 controls physical and/or virtual resources. In some instances, the AASIST program 522 may also embody any combination of the aforementioned elements. To illustrate various aspects of the present disclosure, examples of the AASIST program 522 are presented in which AASIST program 522 includes, without limitation, one or more of: a patient profile transaction, a predicted value estimation profile, an artificial neural network (ANN) request, or a clustering analysis are presented. Additionally, the AASIST program 522 may include other forms of transactions that are known in the art.

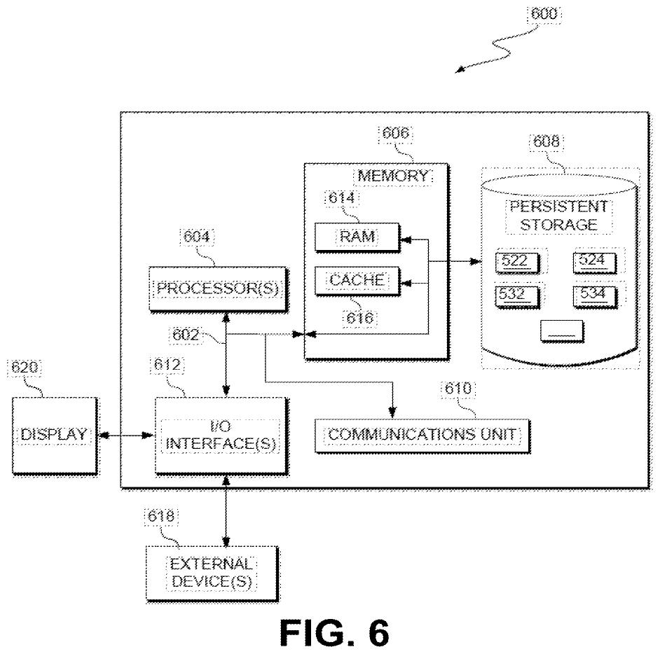

The storage area network (SAN) 530 may be a storage system that includes the server system 532 and the database 534. The SAN 530 may include one or more, but is not limited to, computing devices, server, server-cluster, web servers, databases, and storage devices. The SAN 530 may operate to communicate with the computer system 520 and various other computing devices or computing systems (not shown) over a network, such as network 510. For example, the SAN 530 may communicate with the AASIST program 522 to transfer data between, but is not limited to, the computer system 520 and various other computing devices or computer systems (not shown) that are connected to network 510. The SAN 530 may be any computing device or a combination of devices that are communicatively connected to a local IoT network, i.e., a network comprised of various computing devices including, without limitation, the computer system 520 and various other computing devices to provide functionality described herein. The SAN 530 may include internal and external hardware components as described with respect to FIG. 6. The present disclosure recognizes that FIG. 5 may include any number of computing devices, servers, databases, and/or storage devices, and the present disclosure is not limited to only what is depicted in FIG. 5. As such, in various instances, some or all the features and functions of SAN 530 are included as part of the computer system 520 and/or various other computing devices or computer systems. Similarly, in various instances, some of the features and functions of the computer system 520 are included as part of the SAN 530 and/or another computing device or computer system.

Additionally, in some instances, the SAN 530 represents a cloud computing platform. Cloud computing is a model or service delivery for enabling convenient, on demand network access to a shared pool of configurable computing resources (e.g., networks, network bandwidth, servers, processors, memory, storage, applications, virtual machines, and services) that may be rapidly provisioned and released with minimal management effort or interaction with a provider of a service. A cloud model may include characteristics such as on-demand self-service, broad network access, resource pooling, rapid elasticity, and measured service, may be represented by service models including a platform as a service (PaaS) model, an infrastructure as a service (IaaS) model, and a software as a service (SaaS) model, and may be implemented as various deployment models including as a private cloud, a community cloud, a public cloud, and a hybrid cloud.

In various instances, the SAN 530 may include any number of databases that are managed in accordance with the functionality of an application executing on the SAN 530. In some instances, the database 534 represents the data and the server system 532 represents code that provides an ability to take specific action with respect to another physical or virtual resource and manages the ability to use and modify the data. In other instances, the AASIST program 522 may also represent any combination of the aforementioned features, in which an application executing on the SAN 530 has access to the database 534. To illustrate various aspects of the present disclosure, examples of the application executing on the SAN 530 are presented in which the AASIST program 522 represents one or more of, but is not limited to, a local IoT network and digital media recognition monitoring system.

As shown in FIG. 5, in some instances, the database 534 may be stored on the SAN 530. However, in other instances (not shown), the database 534 may be stored externally and accessed through a communication network, such as network 510, discussed above.

In various instances, the computer system 520 includes the AASIST program 522 which represents an artificial neural network (ANN), wherein the ANN comprises I/O, as well as multiple hidden layers of neurons (i.e., RELU layer). The AASIST program 522 may include a clustering analysis program. The specific elements of the ASSIST program 522 may vary based on its application. For example, the AASIST program 522 may include any number of input, hidden, output layers and various exploratory data and/or statistical data analysis program for executing clustering analysis. In some instances, the AASIST program 522 may analyze the (i) output data of the multilayer neural network and (ii) the change in the weight output, at least in part. The AASIST program 522 may then generate a plurality of optimized data sets of patient parameters based, at least, on a plurality of patient parameters comprising (1) data and (2) metadata. Moreover, the AASIST program 522 may execute a clustering analysis to generate a set of instructions to administer an effective amount of an anti-fibrosis compound, or a pharmaceutically acceptable salt, ester or solvate thereof, to a subject based, at least on, the optimized set of data of patient parameters.

FIG. 6 is a block diagram, 600, depicting an exemplary arrangement of components relating to computer system 520 and SAN 530. It should be appreciated that FIG. 6 provides only an illustration of one implementation and does not imply any limitations regarding the environments in which different embodiments may be implemented. Many modifications to the depicted environment may be made.

As shown in FIG. 6, the computer system 520 and the SAN 530 may include a communications fabric 602, which provides communications between computer processor(s) 604, memory 606, persistent storage 608, a communications unit 610, and input/output (I/O) interface(s) 612. The communications fabric 602 may be implemented with any architecture designed for passing data and/or control information between processors (such as microprocessors, communications, and network processors, etc.), system memory, peripheral devices, and any other hardware components within a system. For example, the communications fabric 602 may be implemented with one or more buses.

The memory 606 and the persistent storage 608 are computer-readable storage media. In general, memory 606 may include any suitable volatile or non-volatile computer-readable storage media. In some instances, memory 606 may include random access memory (RAM) 614 and cache memory 616.

In some instances, the AASIST program 522, the computer interface 524, the server system 532, and the database 534, may be stored in persistent storage 608 for execution and/or access by one or more of the respective computer processors 604 via one or more memories of memory 606. In some instances, the persistent storage 608 includes a magnetic hard disk drive. Alternatively, or in addition to a magnetic hard disk drive, the persistent storage 608 may include a solid-state hard drive, a semiconductor storage device, read-only memory (ROM), erasable programmable read-only memory (EPROM), flash memory, or any other computer-readable storage media that may store program instructions or digital information.

The media used by the persistent storage 608 may also be removable. For example, a removable hard drive may be used for the persistent storage 608. Other examples include optical and magnetic disks, thumb drives, and smart cards that are inserted into a drive for transfer onto another computer-readable storage medium that is also part of persistent storage 608.

Communications unit 610, in these examples, provides for communications with other data processing systems or devices, including resources of the network 110. In these examples, the communications unit 610 includes one or more network interface cards. The communications unit 610 may provide communications by either or both physical and wireless communications links. The AASIST program 522, the computer interface 524, the server system 532, and the client application 534 may be downloaded to the persistent storage 608 through the communications unit 610.

The I/O interface(s) 612 may allow for input and output of data with other devices that may be connected to the computer system 520, the SAN 530, and the client device 540. For example, the I/O interface 612 may provide a connection to external devices 618 such as a keyboard, keypad, a touch screen, and/or some other suitable input device. The external devices 618 may also include portable computer-readable storage media such as, for example, thumb drives, portable optical or magnetic disks, and memory cards. Software and data used to practice embodiments of the present disclosure, e.g., the AASIST program 522, the computer interface 524, the server system 532, and the client application 534, may be stored on such portable computer-readable storage media and may be loaded onto the persistent storage 608 via the I/O interface(s) 612. The I/O interface(s) 612 may also connect to a display 620.

The display 620 provides a mechanism to display data to a user and may be, for example, a computer monitor, or a television screen.

The computer program product may include a computer readable storage medium (or media) having computer readable program instructions thereon for causing a processor to carry out aspects of the present disclosure.

The computer readable storage medium may be a tangible device that may retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein may be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some instances, the electronic circuitry includes, for example, programmable logic circuitry, field-programmable gate arrays (FPGA), or programmable logic arrays (PLA) to execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, may be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that may direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in FIGS. 5-6 illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts or carry out combinations of special purpose hardware and computer instructions.

The programs described herein are identified based upon the application for which they are implemented in a specific embodiment of the disclosure. However, it should be appreciated that any program nomenclature herein is used merely for convenience, and thus the disclosure should not be limited to use solely in any specific application identified and/or implied by such nomenclature.

It is to be noted that the term(s) such as, for example, “Smalltalk” and the like may be subject to trademark rights in various jurisdictions throughout the world and are used here only in reference to the products or services properly denominated by the marks to the extent that such trademark rights may exist.

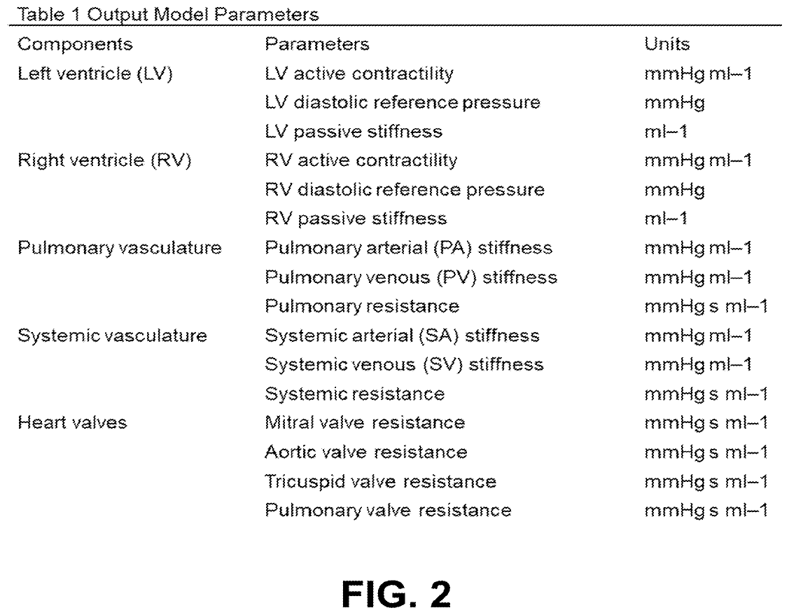

In some instances, the computer-implemented methods may further comprise analyzing data collected using existing diagnostic test techniques. For example, the methods may comprise analyzing data collected using existing diagnostic test techniques to examine heart diseases, including electrocardiogram (ECG), holter monitoring, echocardiogram, exercise or stress test, cardiac catheterization, cardiac computed tomography (CT) scan, and cardiac magnetic resonance imaging. For instance, multiple parameters collected from right heart catheterization (RHC) and trans-thoracic echocardiography (TTE) are used to classify heart failure patients into different groups. Specifically, from TTE measurements, cardiac output (CO) based on the heart rate (HR) may be obtained, the left ventricular out tract flow velocity time integral (LVOT VTI), and the cross-sectional aortic valve area for each patient. RHC is used to measure right ventricular (RV) and pulmonary artery (PA) pressures during systole and diastole along with CO, HR, and pulmonary capillary wedge (PCW) pressure. Quantitative observations were made to reconcile what these clinical datasets describe about the hemodynamics of the right and left sides of the heart and the systemic and pulmonary circulation with a closed-loop model of the cardiovascular system. One of the closed-loop models is TriSeg model of ventricular mechanics incorporating mechanical interaction of the left and right ventricular free walls and the interventricular septum (Lumens, J. et al., Ann. Biomed. Eng. 2009. 37(11): pp. 2234-2255). Various exemplary model parameters are listed in FIG. 2.

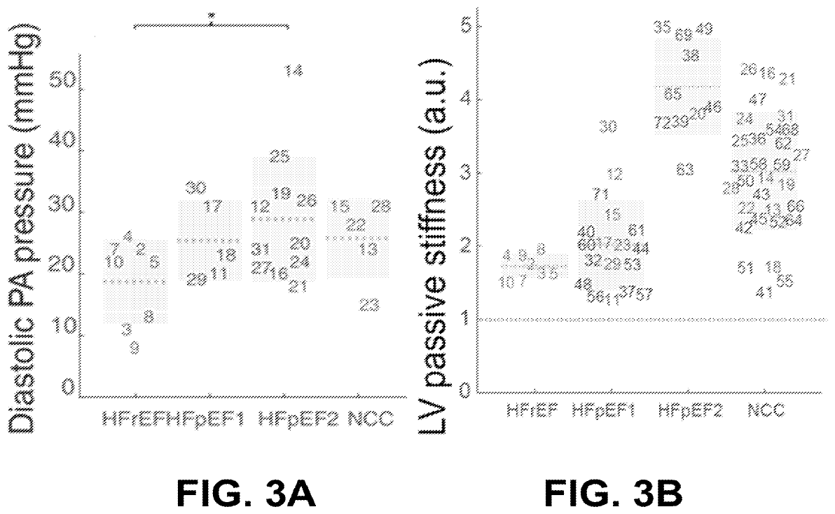

AASIST may be used to analyze RHC and TTE data and impute biomechanical parameters, including diastolic left ventricular (LV) stiffness and contractility by optimizing the output model parameters that best describe personalized cardiovascular phenotypes. There are various approaches to identify the output model parameters matches with a set of input clinical parameters obtained from an individual patient. For example, the genetic algorithm with a population size of 500 and a stall generation limit of 10 generations was used to estimate adjustable model parameters. After analyzing number of HF patients, unsupervised and/or supervised machine learning algorithms classify patients into distinct groups, which trains the artificial intelligence that will be used to classify patients effectively. Examples of using a modified TriSeg model to estimate personalized model parameters with the input parameters are described in published work. This approach was applied to estimate personalized model parameters of 79 patients from their input clinical parameters. For example, the process examined the various heart failure patients' input clinical parameters, including 1) left ventricle (LV) systolic volume, 2) LV diastolic volume, right ventricle (RV) systolic pressure, 3) RV diastolic pressure, 4) pulmonary artery (PA) systolic pressure, 5) PA diastolic pressure, average pulmonary wedge pressure, 6) systemic artery (SA) systolic pressure, 7) SA diastolic pressure, systemic venous pulse pressure, 8) right heart catheter cardiac output, three phenotype groups (phenogroups). While those input clinical parameters of HFpEF patients were indistinguishable, the output model parameter, such as LV passive stiffness formed a group (HFpEF2) with elevated stiffness. In this example analysis, the AI-based classification recognized at least three different phenogroups, including none-classified (FIG. 3). By extending the measurements of RHC and TTE with and without exercise, a series of pressure-volume loops with various inotropic conditions may be estimated and analyzed; therefore, end systolic pressure-volume relationship (ESPVR) and end diastolic pressure-volume relationship (EDPVR) may be determined more effectively and accurately.

Based on clinical diagnostic parameters, AASIST is applied to generate personalized in vitro heart failure disease model that matches with a specific type of heart failure phenotype. The process of generating such disease model is comprised of the following steps:
a. Estimate personalized mechanistic and functional parameters of cardiovascular system components (e.g., LV, RV) using a closed-loop cardiovascular computational model.
b. Train machine learning algorithm with large number of patients.
c. Classify heart failure patients into subgroups based on the mechanistic and functional parameters using machine learning.
d. Produce micro-hearts that recapitulate functional phenotypes of the subgroups by optimizing tissue development conditions. Potential strategies include:
   i. Use cells or tissues isolated from patients in the subgroups.
   ii. Apply disease-inducing factors (e.g., elevated glucose level) identified in blood tests.
   iii. Analyze heart slices if human samples are available.

Using each patient's clinical data, the disclosure first enriches heart failure patients with specific phenotypes through AI-assisted patient stratification technology. The application of closed-loop cardiovascular model or any other mechanistic systems biology models defines tissue mechanics, i.e., stress, strain, and elastic moduli, of cardiovascular system's components such as left and right ventricular myocardium. Defining the mechanical property parameters of tissues enables translating clinical input parameters, including pressures and volumes, to those parameters that may be measured in vitro using cells and tissues isolated or derived from patients. Analyzing large number of patients' clinical data may train the machine learning algorithm to classify patients. A pilot study identified three patient groups (Jones, E. et al., J. Physiol. 2021. 599(22): pp. 4991-5013). In addition, the estimated active contractility and passive stiffness of left ventricle are some of major contributors of classification. Therefore, normalization of those parameters could normalize physiology of cardiovascular system.

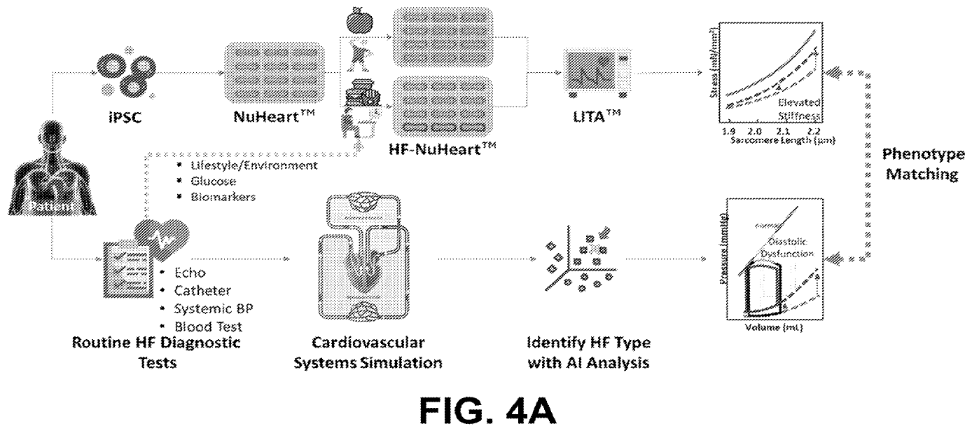

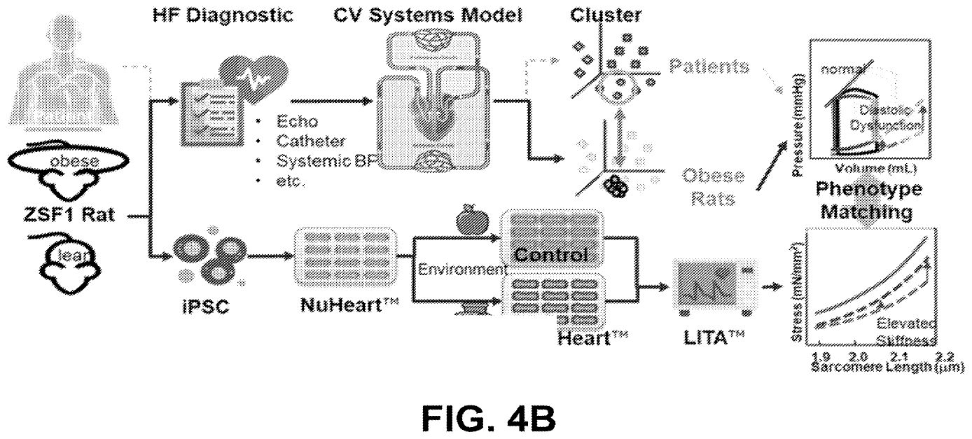

Micro-heart tissues developed using cardiomyocytes and non-muscle cardiac cells, such as fibroblasts and endothelial cells derived from patient-induced pluripotent stem cells (iPSCs) may be used to measure effects of interventions, including small molecules, surgical techniques, electrical pulses, and other potential therapeutic applications on changing the physiological and mechanical properties of micro-heart tissues (FIG. 4A). The mechanical properties of tissue samples (engineered heart tissues or slices isolated from human or animal hearts) are measured by stretching tissues to record their force of contraction and baseline force. The force of contraction represents cardiac systolic contractility and baseline length-force relationship represents stiffness of the samples. Additionally, measurements of action potential, calcium transients, and metabolic activities (e.g., mitochondrial membrane potential, NAD/NADH ratio) provide the excitation-contraction-energy coupling of heart muscle or its equivalent. In addition to using engineered heart, heart slices isolated human donor heart or diseased hearts removed from the patients may be used for the analysis described above.

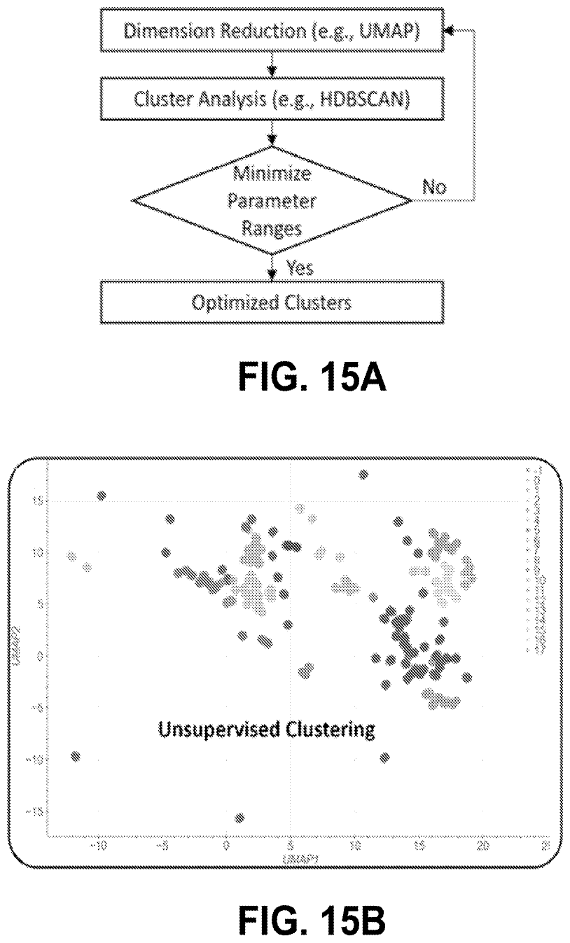

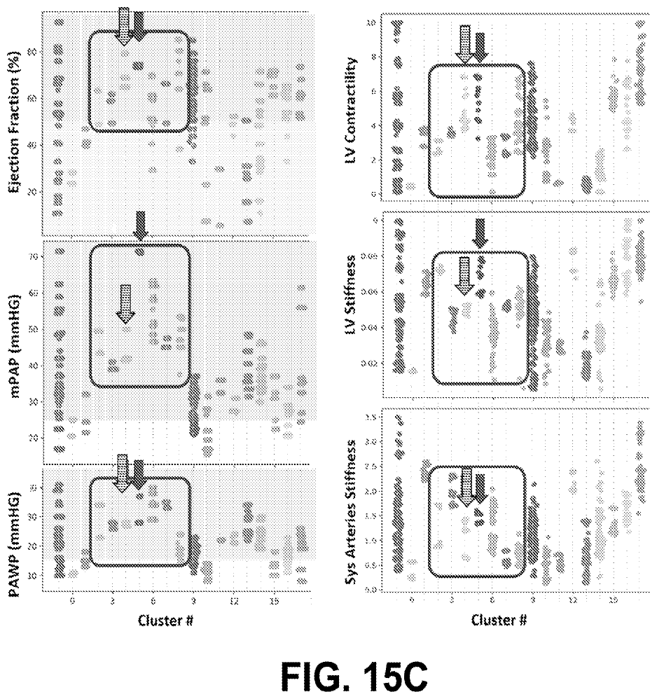

The methods for determining classes of drug candidates to reduce elevated diastolic stiffness, and computer-aided patient stratification methods of identifying a subgroup of heart failure patients generally include the following steps:
a. Determine, e.g., via compound library screening, classes of drug candidates or specific drug candidates to reduce the stiffness of 3D micro tissues made with myofibroblasts (e.g., human cardiac fibroblast cultured with 10% fetal bovine serum). For example, as described in the experimental examples section below, fasudil (hydrochloride hydrate), one of the drug candidates, was expected to reduce elevated diastolic stiffness of left ventricular tissues.
b. Estimate, via computer-aided patient stratification methods, the clinical effects of the drug candidate(s). For example, as described in the experimental examples below, fasudil's clinical effects of improving cardiac reserve and exercise capacity for the patients with highly elevated stiffness parameter of left ventricle was expected to be meaningful. For example, as shown in FIG. 15C, some HFpEF patients have much higher LV stiffness than others. Accordingly, since reduction of LV stiffness is expected upon fasudil administration, fasudil's efficacy among the stratified patients was predicted to be higher than general HFpEF patients.

Based on patients' clinical diagnostic parameters, a similar approach may be applied to identify a personalized in vivo heart failure animal model that matches with a specific type of heart failure phenotype. The process is comprised of the following steps:
a. Estimate personalized mechanistic and functional parameters of cardiovascular system components (e.g., LV, RV) using a closed-loop cardiovascular computational model.
b. Train machine learning algorithm with large number of patients.
c. Classify heart failure patients into subgroups based on the mechanistic and functional parameters using machine learning.
d. Apply a closed-loop cardiovascular computational model to estimate mechanistic and functional parameters of a heart failure animal model.
   i. Compare the estimated parameters of an animal disease model with those of human.
   ii. Identify phenotype group that is best represented by the animal model.
   iii. Produce micro-hearts or heart slices using cells or hearts, respectively, isolated from the animal model.
   iv. Screen library of drug candidates using the in vitro model. Analyze and confirm the effects of drug candidates identified by the in vitro model.

Fasudil inhibits Rho kinase (ROCK). Abnormal ROCK activation was found in elevating vascular tone (J Cardiovasc Dis Res. 2010. 1(4): pp. 165-170). The recent multi-center study PROMIS-HFpEF revealed a high prevalence of coronary microvascular dysfunction (CMD) among about 75% of HFpEF patients (Eur. Heart. J. 2018. 39(37):3439-3450) and the activation of ROCK in CMD is expected. Thus, fasudil expect to normalize abnormal ROCK activity and improve CMD. In addition, FIG. 15C exemplifies various levels of elevated systemic artery stiffness among HFpEF patients. Since fasudil expects to reduce vascular tone, HFpEF patients with abnormally elevated vascular stiffness in combination with elevated LV stiffness would respond to fasudil better than other HFpEF patients. Therefore, in clinicals, efficacy of fasudil to improve hemodynamic parameters and exercise capacity among the stratified patients should be higher than general HFpEF patients.

There are various animal models for heart failure. However, mimicking the heterogeneity of heart failure phenotypes using them, especially inbred animal models, has been challenging. Disease phenotypes measured in each strain are reproducible and exhibit statistical significance in disease indicating parameters compared to their control. Therefore, each animal model should recapitulate a specific disease phenotype that is common in a group of patients. The above process should determine a specific heart failure patient group that is best represented by a specific animal model (FIG. 4B). Then, the approach should improve the translatability of in vivo study to human clinicals.

### B. Closed-Loop Cardiovascular Model

In some instances, to align with individual clinical parameters, a closed-loop cardiovascular model was employed adjusting its parameters to accurately represent each patient's unique cardiovascular state. The model parameters define mechanistic properties of the patient's cardiovascular physiology. Given the complexity of the data, being 11-dimension, a non-linear dimensionality reduction algorithm, including UMAP (Uniform Manifold Approximation and Projection), was used to simplify the data structure. The dimensionally reduced data was then processed using an unsupervised machine learning algorithm, such as DBSCAN (Density-Based Spatial Clustering Application with Noise), to facilitate the identification of distinct clusters.

The goal of clustering patient data is to identify distinct phenotypic groups based on a combination of parameters, e.g., parameters that characterize cardiovascular function. These parameters may be both mechanistic and clinical factors, with a special focus on those that change with the progression of the disease, such as heart failure, and in response to therapeutic interventions. This approach enables the identification of specific patient groups whose parameters may be effectively modulated by targeted therapies.

### V. EXPERIMENTAL EXAMPLES

Without limiting the scope of the instant disclosure, various experimental examples of embodiments discussed above were prepared and the results are discussed below.

### A. Drug Candidate for Heart Failure

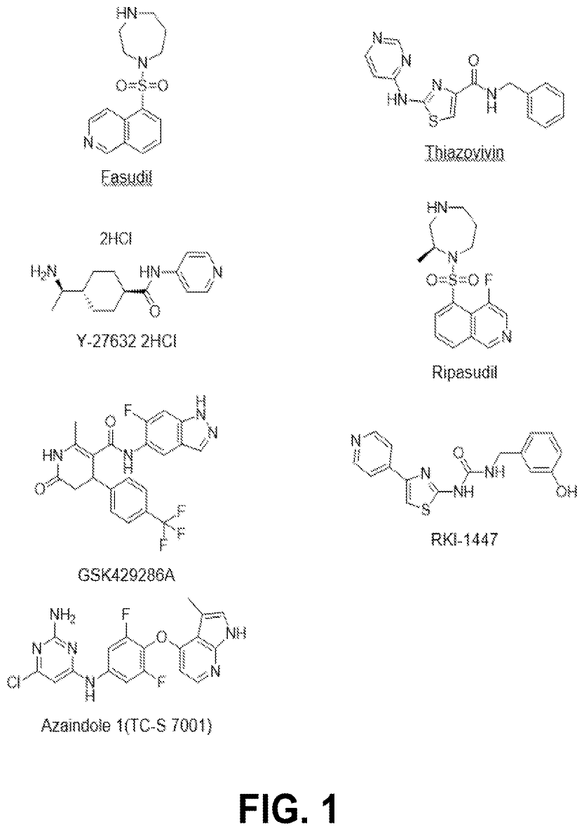

Fasudil (FIG. 1) is a small molecule inhibitor of Rho-kinase (ROCK) that regulates its downstream signal transduction pathways and biological activities, including smooth muscle cells' contractility. InvivoSciences's biomechanical phenotyping assay recorded isometric force by stretching bioengineered tissue-constructs (see, U.S. Pat. No. 10,732,174). The tissue constructs were reconstituted with human fibroblasts that recapitulate connective tissues. Those tissue constructs reconstituted with myofibroblasts recapitulate connective tissue developing fibrosis. By using the assay and tissue constructs, it was observed that fasudil reduced their contractility and tissue stiffness.

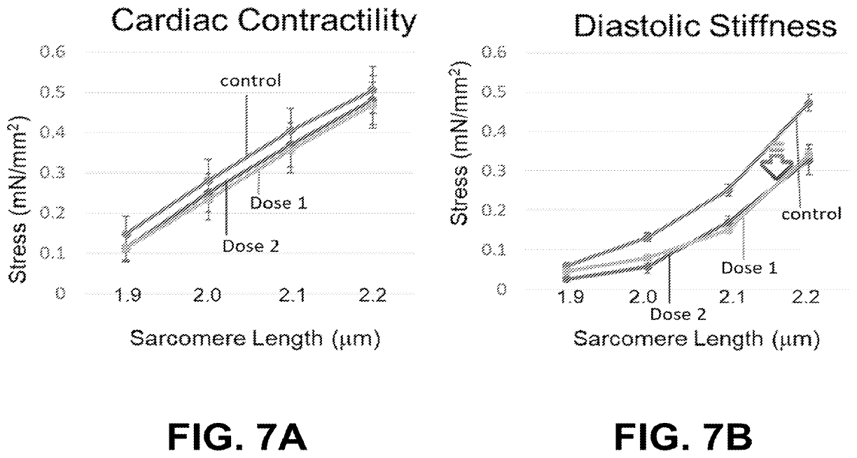

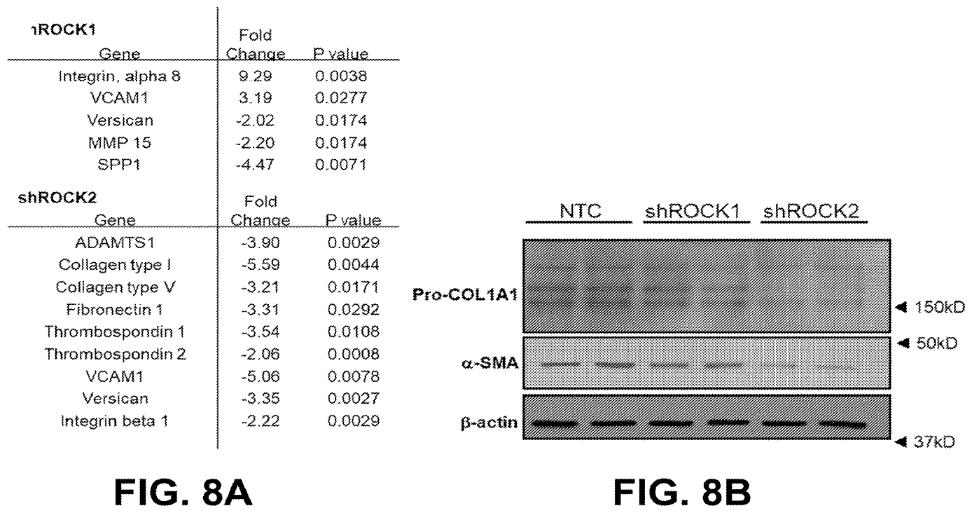

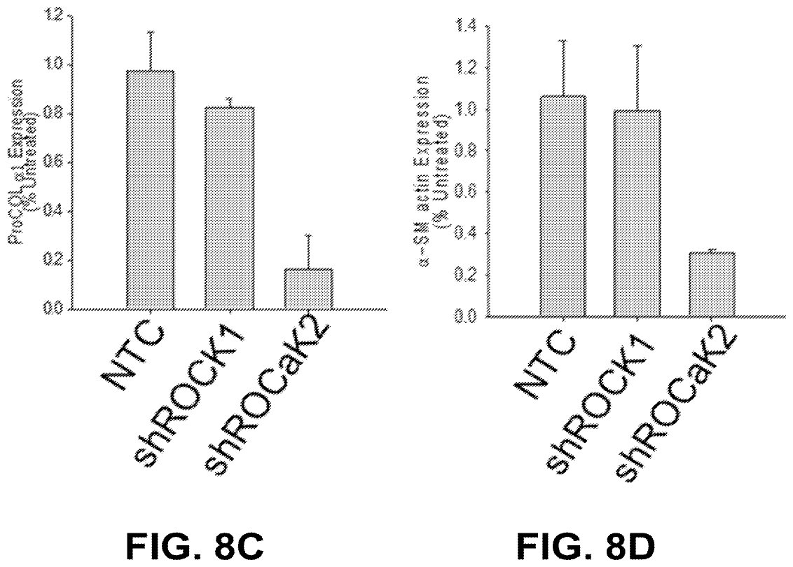

By applying the same biomechanical assay to a micro-heart tissue construct, fasudil treatment reduced the micro-heart tissue construct's stiffness dose-dependently (FIG. 7B). However, fasudil did not change the cardiac contractility of the micro-heart tissues (FIG. 7A). The micro-heart tissue fabrication process produced micro-heart tissues using human cardiomyocytes derived from induced pluripotent stem cells (iPSCs) and non-muscle cells, mainly fibroblasts. Therefore, fasudil is a promising drug candidate to reduce elevated diastolic stiffness of patients with heart failure with preserved ejection fraction (HFpEF). The development of fibrosis in the heart is observed in various types of cardiomyopathies, and heart failure (HF). Myocardial interstitial fibrosis, for example, contributes to left ventricular dysfunction leading to the development of heart failure. Fibrotic myocardium becomes stiffer than healthy tissue to develop diastolic dysfunction. Fasudil targets Rho-kinase (ROCK) that regulates the activity of non-muscle myosin independent of calcium homeostasis. Human cardiac fibroblasts express ROCK1 and ROCK2, which both regulate non-muscle cell contractility similarly. The reduction of ROCK2 had notable effects on the profibrotic gene (FIGS. 7A-B) and protein expression (FIGS. 7A-B). Fasudil treatment on human cardiac fibroblasts showed a similar reduction of αSM expression.

### B. Production of Personalized Fibrotic and Mature Micro-Heart Tissue

Cell types composing the heart include cardiomyocytes (CMs), cardiac fibroblasts (cFBs), endothelial cells (ECs), smooth muscle cells (SMCs), and immune cells (ICs). Human patient- or donor-derived iPSCs are used to produce those cell types applying various differentiation protocols. Many of the differentiation protocols were automated to optimize their cell production yield for mass-production. The patient-derived iPSCs support recapitulating disease phenotypes or sensitivity of developing a specific type of heart failure. The associations between development of heart failures, including hypertrophic and diastolic cardiomyopathies, and genetic mutation(s) are well-established and achieved in databases such as ClinVar. In addition, there are many not well-established links between disease phenotype and genetic backgrounds, which are expected to be validated in the future investigations. Nonetheless, the utility of iPSCs and cells derived from specific patients' group that is classified to develop a certain type of heart failure would be useful to develop in vitro disease models for drug discovery.

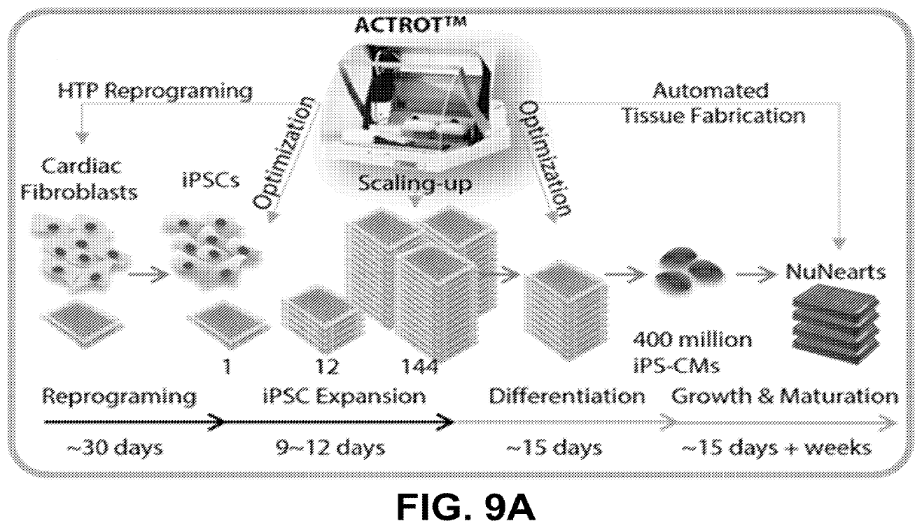

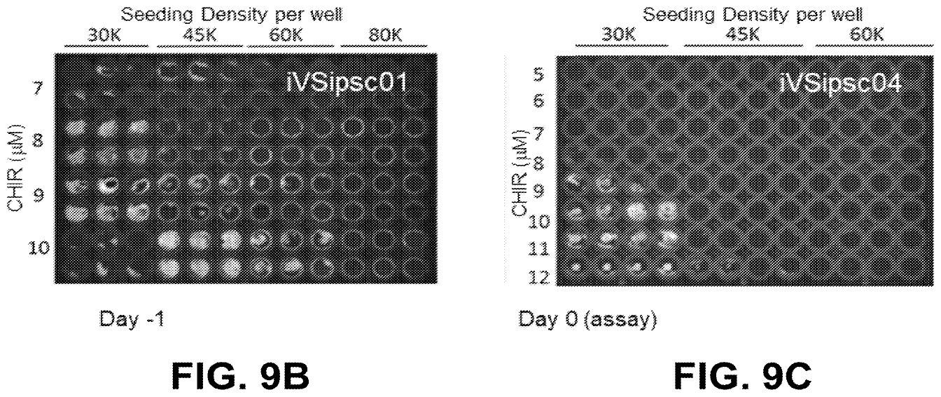

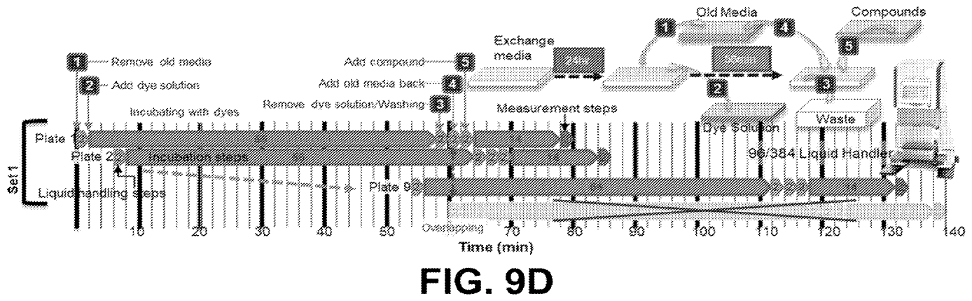

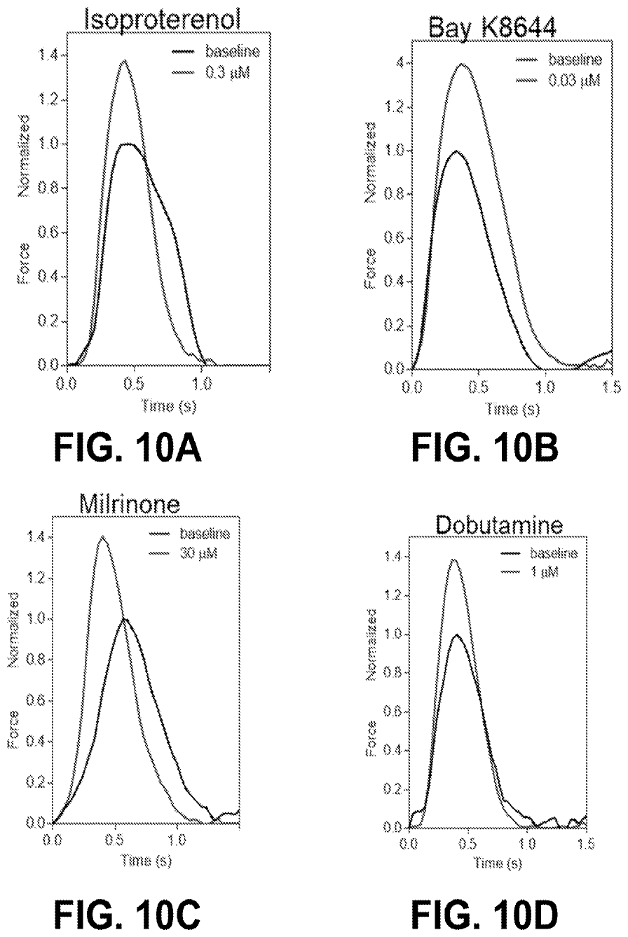

A semi-automated, multi-point quality control (QC) production-process fabricated mature micro-heart tissues in 96-well format. The patient-derived (or gene-edited) iPSCs that were passed the QC test of pluripotency and purity go through the next step. This robust protocol of iPSC differentiation produced cardiomyocytes and non-muscle cells. Various parameters were optimized to produce highly purified (>95%) cardiomyocytes and cardiac non-muscle cells. Various differentiation techniques, a culture robot ACTRO™, and culture media may be used for cardiomyocyte (CM) differentiation and maintenance development and validation. FIGS. 9A-9D show mass-produced micro-heart tissues and their mature phenotype, which were assessed for their response to various positive inotropic and chronotropic agents (FIGS. 10A-10D). To grow mature micro-heart tissue, physiological (15% strain=~2.13 μm sarcomere length) stretching cycles were applied, in addition to constant bi-phasic, low energy (5-10 V, 1 ms duration) electrical stimulation, and maturation medium containing appropriate concentrations and combinations of fatty acids and other factors, including triiodothyronine, dexamethasone, taurine, creatine, and L-carnitine were used.

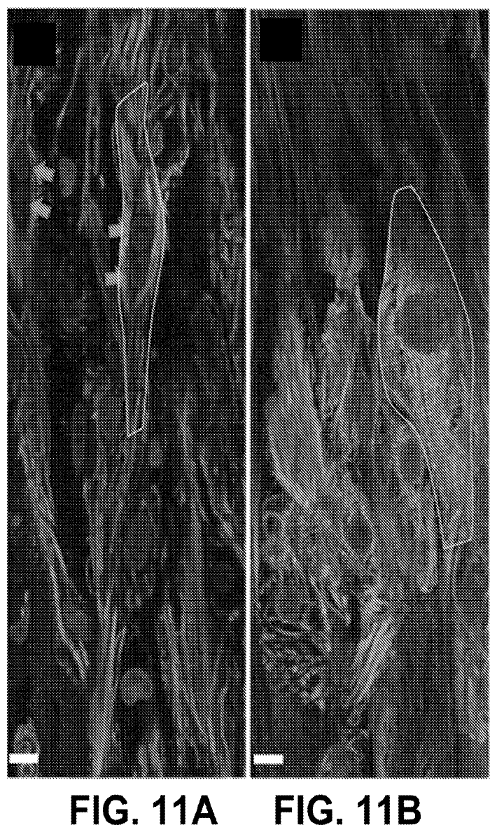

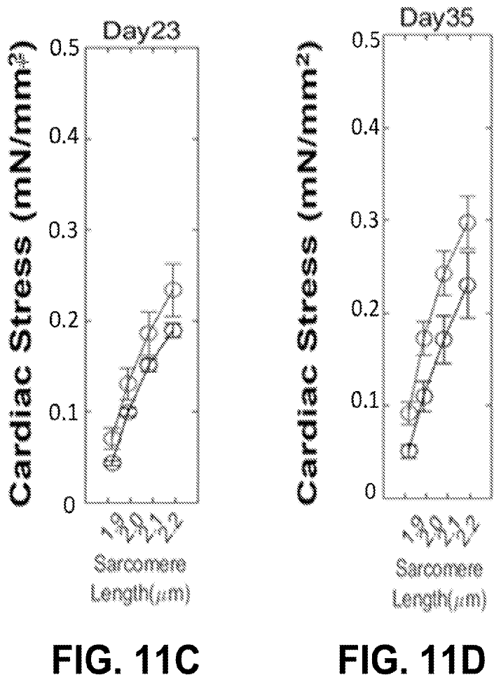

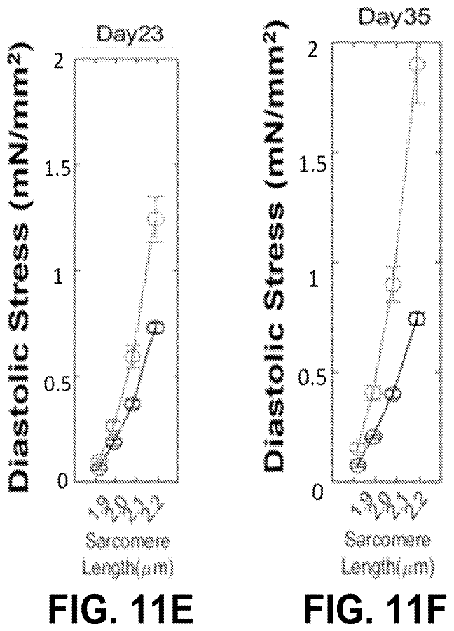

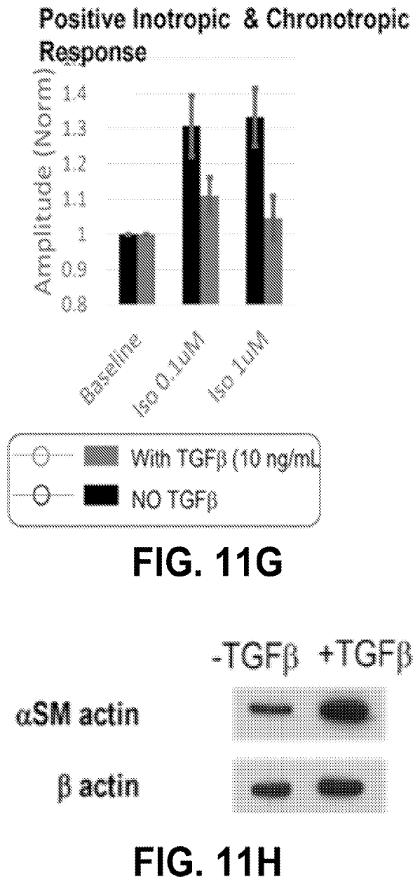

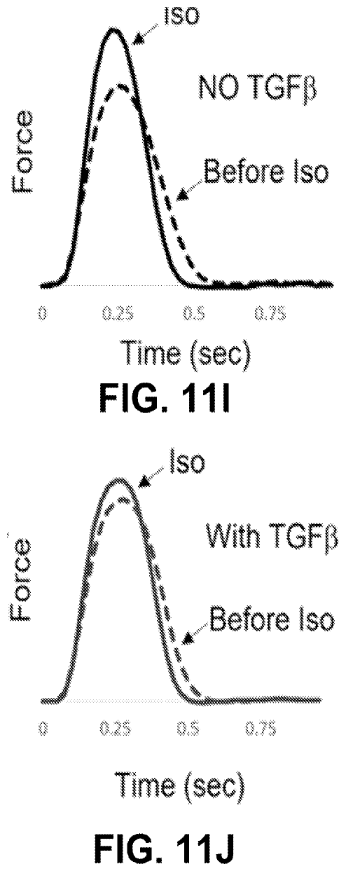

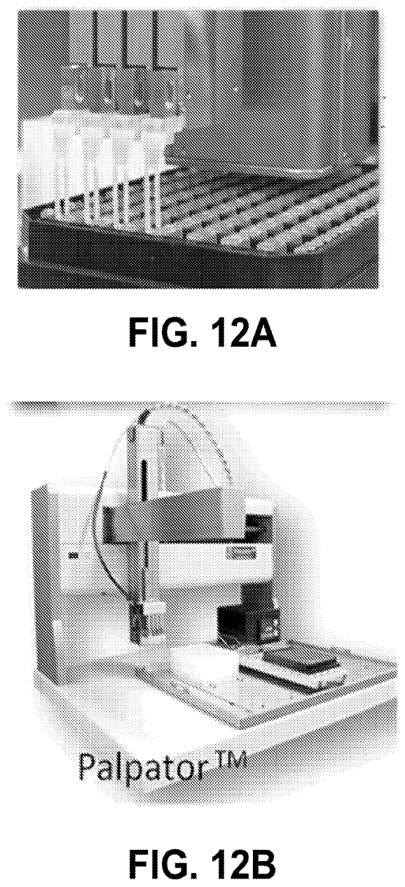

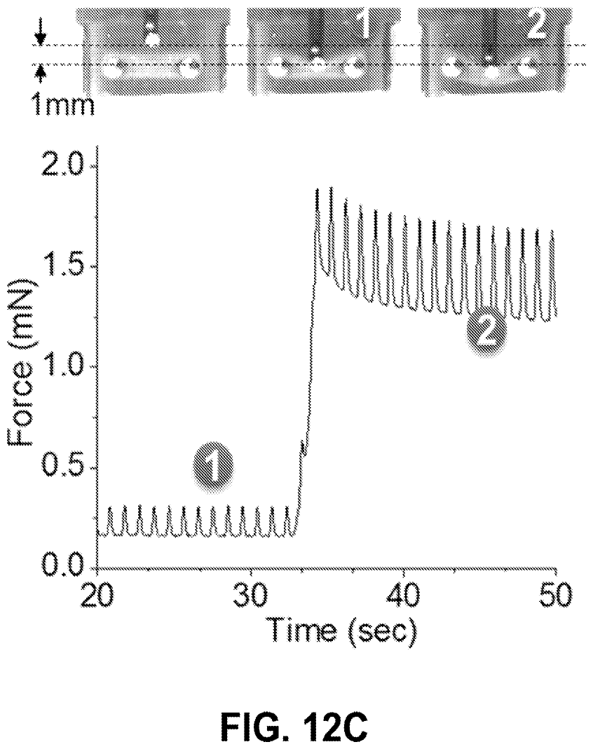

Human micro-heart tissues cultured for 35 days with electrical stimulation matured and loose its spontaneous beating. Myocardial fibrosis is more prevalent in HFpEF than in control, and Transforming growth factor beta (TGFβ) is a major profibrotic factor. A TGFβ-treated micro-heart exhibited active myofibroblasts stained by alpha-smooth muscle (αSM) actin antibodies (FIGS. 11A-J), and its diastolic stress (i.e., stiffened) and cardiac contractility are increasing. The mature micro-heart tissues exhibit positive inotropic and chronotropic responses after isoproterenol (ISO) treatment. A chronic TGFβ treatment, however, impaired the ISO-induced positive inotropic and chronotropic responses (FIG. 10). Western blot analysis showed increased expression of αSM actin. The data suggest that TGFβ-treatment-induced samples develop 1) diastolic stiffening, 2) hyper cardiac contractility, and 3) loss of cardiac contractile reserve (impaired responsiveness to inotropy and chronotropic stimuli). The elevated diastolic stiffness and loss of contractile reserve are common in HFpEF patients. Some HFpEF patients, including hypertrophic cardiomyopathy, show a hypercontractile phenotype. A TGF-β signaling pathway in the pathogenesis of diabetes mellitus (DM) is well-known. Moreover, in rat models, the presence of diabetes led to the induction of a fetal gene program, typically seen in the setting of TGF-β over-expression and hypertrophy. Therefore, human induced pluripotent stem cell (iPSC) derived micro-heart tissue (NuHeart™ micro-heart tissue) will be used with TGFβ-treatment as a base of the proposed studies.

### C. Mechanical Assays

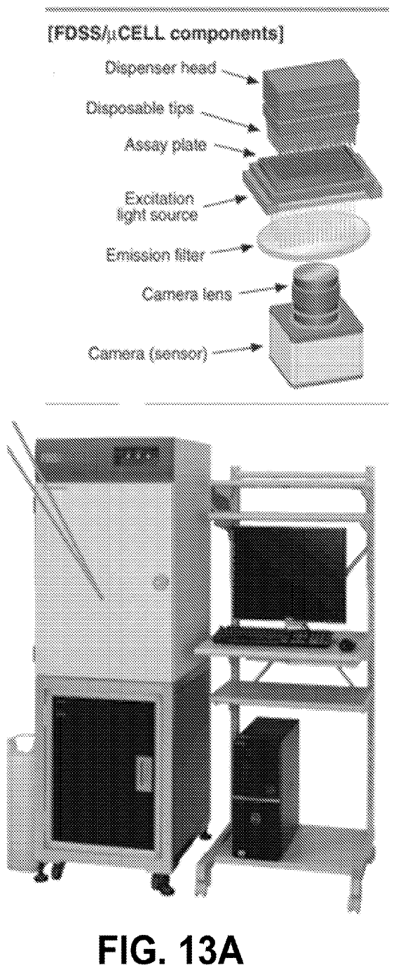

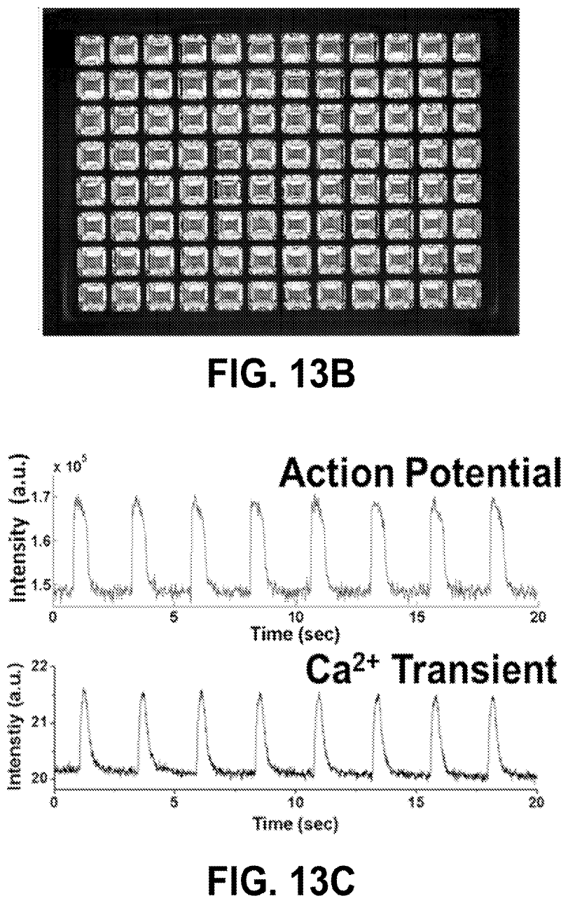

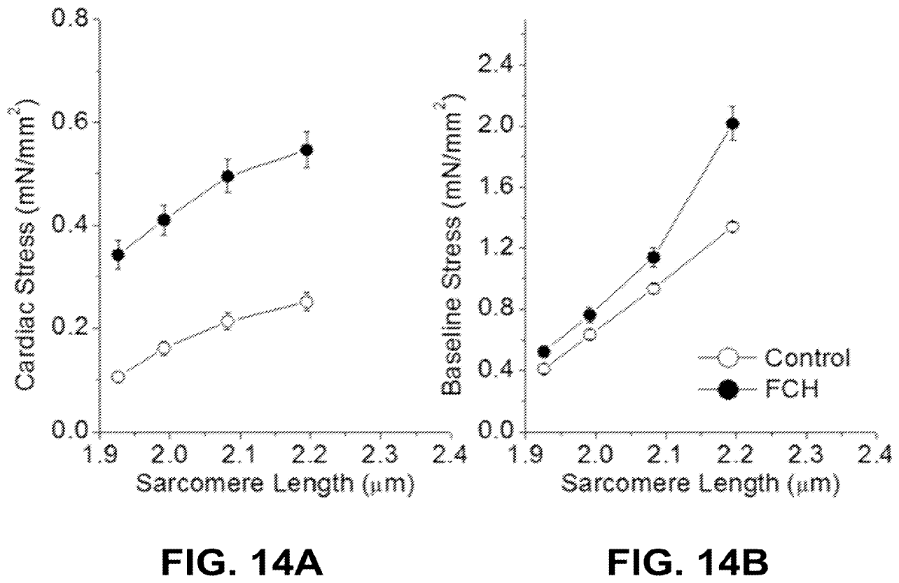

A force probe stretching approach was used to record the length-tension relationship to analyze cardiac contractility and stiffness of micro-heart tissue. Micro force-transducers (FIG. 11) of the mechanical assay device, Palpator™, measured resistance forces that were required to stretch micro-heart tissue growing in 96-well plates. As the probe moved down by a motorized arm, samples' cardiac contractile and baseline forces were measured at different tissue lengths. Sarcomere length was calibrated to strain (% of stretch over original sample length) according to a published protocol (Asnes, C. F. et al. Biophys. J. 2006. 91(5): pp. 1800-1180). (Fourier analysis of sarcomere patterns in fixed micro-heart tissue). In addition, other biological indicators such as calcium transients and action potential are measured using biological indicator dyes and high throughput microplate readers (FIGS. 13A-C). For example, micro-heart tissue with cardiomyocytes derived from gene-edited iPSC-derived cardiomyocytes was fabricated with a cardiac troponin T R92Q mutation (familial hypertrophic cardiomyopathy, i.e., “FCH”). This hypertrophic cardiomyopathy (HCM) is known to exhibit a hypercontractile phenotype in humans, and hypercontractile phenotype of FCH (familial hypertrophic cardiomyopathy) microtissue was recognized by the increase cardiac stress in length tension plot (FIGS. 14A-B). In addition, micro-heart tissue had an increased diastolic stiffness, also known to be seen in hypertrophic cardiomyopathy.

### D. Stratification of HFpEF Patients

Physiological and pathological heterogeneity of HFpEF have been serious challenges to validate the efficacies of drug candidates in clinical trials, especially in large cohort Phase III trials. Classified HFpEF patients were stratified into distinct groups by an AI-assisted clustering of diagnostic parameters.

### E. Diagnostic Parameters

The stratification technology first analyzed well-defined and widely accepted diagnostic parameters obtained using various modalities such as echocardiogram, magnetic resonance imaging, and cardiac catheters. Those parameters included systolic and diastolic right ventricular pressure, systolic and diastolic pulmonary arterial pressure, average pulmonary capillary wedge (PCW) pressure, systolic and diastolic systemic pressure, heart rate during the catheterization, cardiac output (CO), body weight, height, and sex.

To measure those parameters, for example, a Swan-Ganz catheter may be inserted through the subject's jugular vein to measure the pressure at the tip of the catheter as it is advanced into the pulmonary artery. Besides pressure information, CO was estimated by using the thermodilution or Fick methods (JAMA Cardiol. 2017. 2(10): pp. 1090-1099). The thermodilution technique estimated CO by measuring dispersion of a cold saline bolus injected at the proximal end that is then sensed at the distal end of the catheter. The Fick method measured venous and arterial oxygen saturation a given whole body oxygen consumption (\dot{V}O₂) based on weight, height, and sex.

A transthoracic echocardiogram (TTE) was one of the modalities that was used to analyze chamber volumes including left ventricular volume in systole and diastole and heart rate. The left ventricular volumes in systole and diastole were measured as either a single diameter across the left ventricle just below the mitral valve leaflet tips or volumes quantified from tracings of the left ventricle from apical two- and four-chamber views. The single diameter derived volumes assumed the left ventricle may be approximated as a truncated prolate spheroid with a nonlinear relationship between the diameter and length of the ventricle. Volumes derived from the two- and four-chamber views were calculated by the method of discs, also known as Simpson's method. A cardiologist estimated when the quality of the image allowed using Simpson's method, determined the heart rate, and extracted a left ventricular outflow tract velocity time integral (LVOT VTI) estimating CO. Cardiac magnetic resonance (CMR) imaging is the gold standard approach to measure ventricular volumes and fibrosis. When necessary, the analysis may also implement CMR.

### F. The Cardiovascular Systems Model

Various closed-looped cardiovascular models have been introduced. Depending on the computational resources and number of available data to define input parameters for the model, the model complexity will be determined. The pericardial compartment may be removed and the zero pressure (or dead space) volumes in all vascular and ventricular compartment were set to zero. A factor was added to recruit stressed blood volume from the nominal value of 30% total blood volume, which has been posited to occur in heart failure (Fudim, M. et al. J. Am. Heart Assoc. 2017, 6(8), e006817). However, no volume was recruited to represent any of the HFpEF or HFrEF patients in this study. Equations for the reduced cardiovascular system model used in this study are given in a previous publication (Smith, B. W. et al., Med Eng. Phys. 2004. 26(2): pp. 131-139), however, other types of formulation to represent closed-loop cardiovascular system may be employed.

The example model had 16 model parameters (FIG. 2) which were adjusted during the optimization process. Parameter descriptions for the 16 model parameters are shown in Table 1 (FIG. 2). To define some of the parameters, the example analysis adhered to the following process: 1) left ventricular elastances was calculated from measured volume and estimated pressure in systole; 2) left ventricular diastolic stiffness was calculated from measured volume and estimated pressure in diastole; 3) right ventricular elastances were calculated from measured pressure and estimated volume in systole; 4) right ventricular diastolic stiffness was calculated from measured pressure and estimated volume in diastole; 5) systemic elastances were calculated from measured pulse pressures for the arterial compartments, estimated pulse; 6) pressures for the venous compartments, and estimated stressed volumes were determined; 7) pulmonary elastances were calculated from estimated and measured pulse pressures and estimated stressed volumes; 8) systemic and pulmonary resistances were calculated from measured systemic and pulmonary average arterial pressures and estimated systolic venous pressure along with the measured right heart catheterization (RHC) cardiac output. Total blood volume was calculated based on the height, weight, and sex of each patient as described in a previous study looking at heart transplant patients, utilizing a known expression method (Nadler, S. B. et al., Surgery. 1962. 51(2): pp. 224-32). The initial distribution of stressed and unstressed blood volume among the six vascular compartments was based on the work by Beneken (Reeve, B. M. et al., American Heart Journal. 1968. 75(3): pp. 432-433) in which a total stressed volume of 18.75% was assumed.

### G. Optimization of Parameters

For each patient, the computational analysis estimated the adjustable parameters by minimizing the least square error between the simulations and data for a set of estimating parameters: right ventricular pressure in systole and diastole, pulmonary artery pressure in systole and diastole, average pulmonary capillary wedge pressure, systemic artery pressure in systole and diastole, CO during RHC, left ventricular volume in systole and diastole, and CO during TTE.

Since the heart rate during RHC and TTE may be different, two separate simulations were run—one simulating the RHC and one simulating the TTE; however, both simulations were run with one set of parameter values with the assumption that the parameters representing cardiac function do not change appreciably across procedures for a single patient. Values of the clinical measures were calculated over the cardiac cycle after the system reached a steady state of pulsatile pressures and flows.

This was assured by allowing the simulations to run for 50 beats. Once this steady state was reached, the maximum and minimum values of the pressure and volume data of the last 5 beats were used to compute the total residual error. The capillary wedge pressure and the CO represents average values over the cardiac cycle; therefore, their values were averaged over the cardiac cycle before being compared to the TTE and RHC measures. Estimates for the adjustable parameters were obtained using a genetic algorithm optimization implemented in MATLAB™

### H. Artificial Intelligence

After optimization and determination of model parameters that represent individual model, three different clustering techniques grouped individuals into a population based on similar characteristics. The clinical data and optimized parameter values were compiled into separate matrices, 0 and 1, respectively, where each row represents a given patient and each column represents a clinical measure or optimized parameter value. Before any of the clustering methods was applied, each column was centered by subtracting the average of each column from each element in that column. Clinical data and optimized parameters with different units were normalized by dividing each variable by its standard deviation. After performing the principal component analysis (PCA) to identify principal components of the multi-dimensional data, the HFpEF stratification algorithm used various clustering techniques, including k-means and hierarchical clustering, to identify distinguishable groups. This approach has been termed “AI-Assisted, Systems-biology Integrated patient Stratification Technology (AASIST)” for identifying a specific group of HFpEF patients.

Increasing the number of patients expanded training data to train the stratification algorithm, AASIST, which enabled differentiating groups of HFpEF patients with a greater confidence. This training was be achieved by analyzing existing data in the electronic health record data base. A well-trained AASIST groups HFpEF patients based on their cardiovascular function. The application of AASIST on HFpEF patient data demonstrated that left ventricle active contractility and passive stiffness are significantly different in groups clustered by the analysis described above. Fasudil reduced the passive stiffness of human micro-hearts derived from induced pluripotent stem cells. Therefore, it is expected that fasudil treatment or other Rho-kinase inhibitor (ROCK) treatment may reduce the elevated passive stiffness of HFpEF patients stratified the AASIST. The efficacy of fasudil and other ROCK inhibitors should be significant if patients are selected with the AASIST.

### I. Analysis of Gene Expression

Cardiac amyloid, including monoclonal immunoglobulin (AL amyloid) and transthyretin (TTR amyloid), deposition may cause HFpEF. Screening of HFpEF patients estimated ~13% of the HFpEF cases admitted to the hospital was confirmed to exhibit TTR in the biopsy sample and ~17% of autopsy samples among the patients with antemortem diagnosis of HFpEF had deposition of TTR in myocardium. In general, histological detection of amyloid deposits on tissue biopsy specimens is the only diagnostic approach for amyloidosis. Exceptionally, the cardiac transthyretin amyloidosis may be diagnosed without a tissue biopsy if stringent criteria are met. Because tafamidis has been approved recently to treat the cardiac transthyretin amyloidosis, cardiologists perform diagnosis testing to detect amyloid deposition.

According to cardiologists, nearly 50% of biopsy samples are negative of amyloid. However, those HFpEF patients, including amyloid positive, show elevated plasma levels of N-terminal pro-B-type natriuretic peptide (NT-pro BNP>~2,000 ng/L), indicating their advanced stage of HF. It is not clear whether the levels of NT-pro BNP correlate with a degree of cardiac fibrosis. Gene expression studies of HFpEF patients with elevated NT-pro BNP without amyloid deposition showed a unique gene expression pattern related to the signal transduction pathways, including MAPK signaling, Hippo signaling pathway, and regulation of actin cytoskeleton (Hahn, V.S. et al., Circulation. 2021, 143(2): pp. 120-134.). Those signaling pathways regulate signaling pathways that regulate fibrosis. Therefore, a Rho-kinase inhibitor (e.g., fasudil) or other anti-fibrosis compound treatment may be administered reduce fibrosis. During the further gene expression analysis of biopsy samples, a panel (1-10) of genes that may differentiate this group of patients that will respond to the fasudil treatment.

### J. Combination of AASIST and Gene Expression Analysis

The HFpEF patients stratified using AASIST and gene expression analysis should overlap in general. However, both parameters will be used to narrow the HFpEF patients to improve efficacy of treatment in clinical trials.

K. Inhibitors targeting Rho-Kinase:

As shown in FIG. 1, the inhibition of Rho-kinase (ROCK) activity has similar pharmacological effects as fasudil. Other small molecules inhibitors targeting Rho-kinase, including Thiazovivin, Y-27632 2HCI, GSK419286A, Ripasudil, RKI-1447, Azaindole 1 (TC-S 7001), have similar pharmacological properties including the efficacy to treat cardiac fibrosis.

### L. Closed-Loop Cardiovascular Models

Various types of Closed-Loop cardiovascular models may be used. Described below are various non-limiting examples of Closed-Loop cardiovascular models.

### 1. Windkessel Models

Windkessel models are the simplest form of cardiovascular models. These models use electrical circuit analogies to represent the compliance and resistance of blood vessels, particularly for modeling blood pressure and flow in large arteries. Windkessel models typically focus on the arterial system.

### 2. Lumped Parameter Models:

Lumped Parameter models divide the cardiovascular system into segments, each characterized by its resistance, compliance, and inertance. Since these models provide a more detailed representation than Windkessel models, Lumped Parameter models may be used to study the dynamics of the entire cardiovascular system.

### 3. Distributed Parameter Models

Distributed Parameter models consider the spatial variations along blood vessels. These models use partial differential equations to represent blood flow and pressure, making them more complex and detailed than lumped parameter models. Distributed Parameter models may be particularly useful for studying wave propagation phenomena in arteries.

### 4. 0D, 1D, 2D, and 3D Models

0D, 1D, 2D, and 3D models are numerical models that vary in their spatial dimensionality. For example, 0D (zero-dimensional) models are similar to Lumped Parameter models, and 1D models represent the axial variation in blood vessels. Moreover, 2D (two-dimensional) and 3D (three-dimensional) models provide detailed representations of blood flow and vessel wall dynamics and may be used in computational fluid dynamics (CFD) studies.

### 5. Patient-Specific Models

Patient-specific models are tailored to the specific cardiovascular physiology of individual patients, often using medical imaging data. These models may be used in personalized medicine for diagnostic and therapeutic purposes.

### 6. Multi-Scale Models

Multi-scale models integrate different levels of physiological detail, from cellular and tissue levels up to the whole organ and system levels. These models may be used to study the interactions between various scales, such as how molecular and cellular changes may affect overall cardiovascular function.

### 7. Hybrid Models

Hybrid models combine elements of both Lumped Parameter and Distributed Parameter models or combine computational fluid dynamics (CFD) with structural mechanics models (fluid-structure interaction). They are used for more complex simulations where different aspects of cardiovascular physiology need to be integrated.

To align with individual clinical parameters, a closed-loop cardiovascular model was employed, as described above, adjusting its parameters to accurately represent each patient's unique cardiovascular state. The model parameters define mechanistic properties of the patient's cardiovascular physiology. Given the complexity of the data, being 11-dimension, a non-linear dimensionality reduction algorithm, including UMAP (Uniform Manifold Approximation and Projection), was used to simplify the data structure. The dimensionally reduced data was then processed using an unsupervised machine learning algorithm, such as DBSCAN (Density-Based Spatial Clustering Application with Noise), to facilitate the identification of distinct clusters.

As described above, the goal of clustering patient data is to identify distinct phenotypic groups based on a combination of parameters that characterize cardiovascular function. These parameters may be both mechanistic and clinical factors, with a special focus on those that change with the progression of heart failure and in response to therapeutic interventions. This approach enables the identification of specific patient groups whose parameters may be effectively modulated by targeted therapies, e.g., treatment with a small molecule inhibitor of Rho-kinase (ROCK). A specific case for fasudil is described below.

The algorithms employed, UMAP (Uniform Manifold Approximation and Projection) and DBSCAN (Density-Based Spatial Clustering Application with Noise), were configured with several adjustable parameters to optimize data structuring and clustering. A cost function, defined as the range of selected parameter values in each cluster, was established. These parameters include left ventricle contractility, left ventricle stiffness, systemic artery stiffness, mean pulmonary artery pressure (mPAP), and pulmonary artery wedge pressure (PAWP). The optimization process aims to minimize this cost function, facilitating the identification of clusters characterized by minimal variability in their parameter value distribution.

The analysis depicted in FIGS. 15A-C illustrate an exemplary heart failure (HF) patient stratification. Phenotypes of HF patients (n=137) were stratified into 19 pheno-groups defined by the combinations of mechanistic and clinical parameters. As described, HF patients who underwent invasive RHC are presumably enriched with those with PH. Then, as RHC parameters were analyzed, such as mean pulmonary arterial pressure (mPAP) and pulmonary artery wedge pressure (PAWP), data include ejection fractions being lower or higher than 50%, but many had mPAP>25 mmHg and PAWP>15 mmHg. According to the classical diagnostic range, those patients may be classified as PH with HFpEF. LHD, including both HFrEF and HFpEF, is one of the most common contributors to PH and its prevalence in LHD is estimated between 23% and 80% depending on the analysis (Al-Omary, M.S., et al., Hypertension, 2020. 75(6): pp. 1397-1408). It also showed that among HFpEF patients, an increased left-sided filling pressure may lead to a combined pre-capillary and post-capillary (Cpc) phenotype, causing pulmonary arterial vasoconstriction and remodeling. Patients classified by RHC data with mPAP≥25 mmHg and PAWP>15 mmHg align with trends noted in literature (Dixon, D. D., et al., Heart Failure Reviews, 2016. 21(3): pp. 285-297). Like the literature, in the analysis, such patients with RHC data with mPAP≥25 mmHg and PAWP>15 mmHg were found in both reduced and preserved ejection fraction categories, but predominantly in HFpEF.

To illustrate the phenotypic stratification approach, the analysis depicted in FIG. 15C focused on two HFpEF groups (Group 3 and 4), indicated by solid and striped arrows in FIG. 15C. Both groups exhibited comparable LV contractility and systemic arterial stiffness yet differ in LV stiffness. This preliminary analysis, based on a limited dataset, suggests potential for identifying diverse patient subgroups within traditional phenotyping and pinpointing a target population. Specifically, patients indicated in solid arrow could benefit from fasudil treatment by higher LV stiffness reduction than other groups by affecting myofibroblast contractility in the left ventricle.

### M. Benefit of Clustering in Enrichment of Clinical Study Populations

The patient stratification process is particularly advantageous for targeted therapies that are known to modify specific cardiovascular parameters. For instance, consider an antihypertensive drug designed to reduce systemic arterial stiffness. By conducting a clinical study enriched with patients who have significantly elevated systemic arterial pressure, it may be expected that statistically significant results will be achieved with a smaller patient cohort than without such enrichment. In the case of fasudil, which is expected to reduce multiple parameters including left ventricle (LV) contractility, LV stiffness, and systemic artery (SA) stiffness, the clinical study should specifically select patients exhibiting elevations in all these parameters, as opposed to those in different clusters.

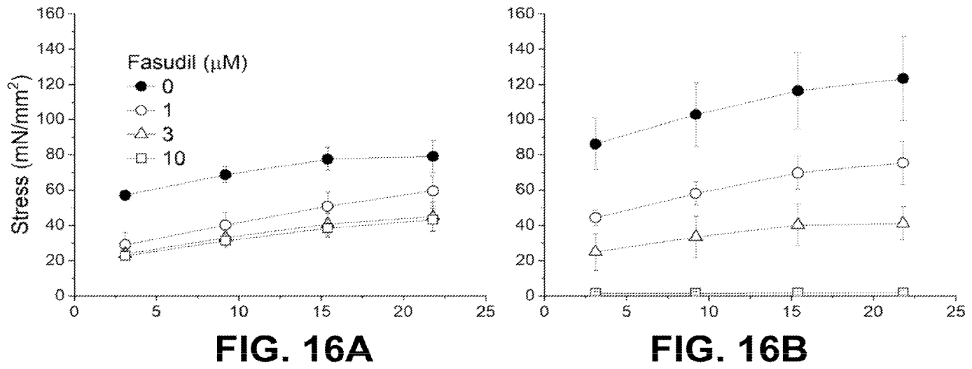

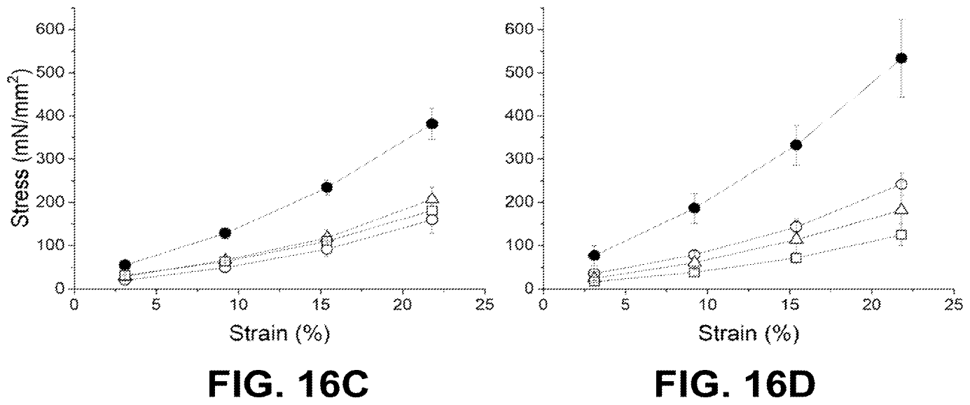

To exemplify this patient stratification process, engineered heart tissues (EHTs) were prepared, the tissues containing cardiac fibroblasts (10% of cardiomyocytes at fabrication) derived from human induced pluripotent stem cells (iPSCs). Five days post-fabrication, one half of the engineered heart tissues (EHTs) was cultured regularly (FIG. 16A and FIG. 16C), and the other half was treated with 1 ng/mL TGFβ (FIG. 16B and FIG. 16D). Since the EHTs contained cardiac fibroblasts (10% of cardiomyocytes at fabrication) derived from human induced pluripotent stem cells (iPSCs), TGFβ-treated EHTs exhibited increased passive stiffness. The effect was clearly represented in the elevated passive stress-strain curve and its slope represented by the closed circles (0 μM fasudil data points) in FIG. 16C and FIG. 16D.

After 15 days, the EHTs, both with and without TGFβ treatment, were administered fasudil at concentrations of 1, 3, and 10 M (FIGS. 16A-16D). Notably, the plasma concentration of fasudil in intravenous formulations was approximately 0.36 μM (Satoh, S., et al., Life Sci, 2001. 69(12): pp. 1441-1453). Therefore, the higher dose, especially 10 M, might be too high and substantially reduce cardiac contractility in TGFβ-treated EHTs by day 29. Nonetheless, fasudil effectively reduced the passive stiffness of EHTs, both with and without TGFβ treatment, though more pronounced effect was observed for the TGFβ-treated tissues. Furthermore, the impact of fasudil on cardiac contractility was less significant one day after treatment (data not shown). However, by day 29, fasudil reduced cardiac contractility in EHTs, irrespective of TGFβ treatment.

In summary, short-term administration of fasudil (less than 24 hours) effectively reduced passive stiffness in EHTs without significantly affecting cardiac contractility. In contrast, long-term administration (3-14 days) of fasudil was found to reduce both cardiac contractility and passive stiffness in EHTs. The mechanisms underlying these effects involve fasudil-induced Rho kinase inhibition. Although Rho kinase inhibition may directly reduce cardiac contractility, Rho kinase inhibition has previously shown minimal impact on cardiac contractility, particularly in human ventricular muscle strips (Grimm, M. et al., Cardiovasc Res, 2005. 65(1): pp. 211-220).

These results suggest a mechanical interconnection between the cardiac contractility unit and its nonlinear passive elastic components, both in parallel and in series. The reduction in passive stiffness may be attributed to decreased myofibroblast contractility. Moreover, the reduction in the passive mechanical element appears to diminish cardiac contractility, considering that this component somewhat amplifies cardiac contractility by elevated myofibroblast's contractility and fibrotic remodeling of extracellular matrices.

From a clinical standpoint, the therapeutic benefits of fasudil are likely to be most pronounced in heart failure patients characterized by increased diastolic stiffness and cardiac contractility; for instance, a group indicated by a solid arrow in FIG. 15C. Additionally, due to its ability to reduce smooth muscle contractility, fasudil shows promise as a treatment for vascular and pulmonary hypertension. Targeting heart failure patients who specifically display the conditions most responsive to fasudil could enhance treatment efficacy. This approach of patient enrichment is valuable for identifying individuals who are most likely to benefit from fasudil. Furthermore, employing a similar strategy of patient enrichment could also enhance the efficacy of clinical trials for various other targeted therapies.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:
Clause 1. A method of treating heart failure with preserved ejection fraction (HFpEF), the method comprising:
   administering a therapeutically effective amount of a Rho-kinase inhibitor, an anti-fibrosis compound, or a pharmaceutically acceptable salt, ester, or solvate thereof, to a subject experiencing:
      diastolic dysfunction,
      cardiac fibrosis,
      pulmonary hypertension, and
      a left ventricular ejection fraction of ≥50%,
wherein the subject does not have cardiac amyloid deposits.
Clause 2. The method of clause 1, wherein the Rho-kinase inhibitor is:

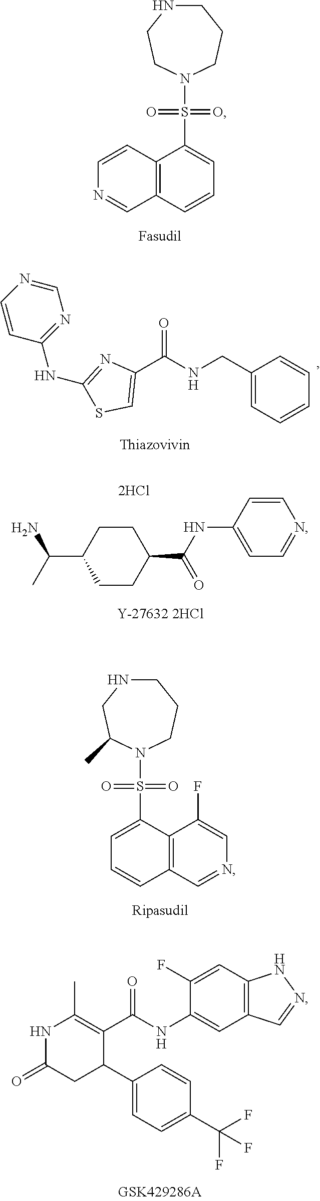 [SMILES: O=S(=O)(c1cccc2cnccc12)N1CCCNCC1 ; O=C(NCc1ccccc1)c1csc(Nc2ccncn2)n1 ; C[C@@H](N)[C@H]1CC[C@H](C(=O)Nc2ccncc2)CC1 ; C[C@H]1CNCCCN1S(=O)(=O)c1cccc2cncc(F)c12 ; CC1=C(C(=O)Nc2cc3cn[nH]c3cc2F)C(c2ccc(C(F)(F)F)cc2)CC(=O)N1]

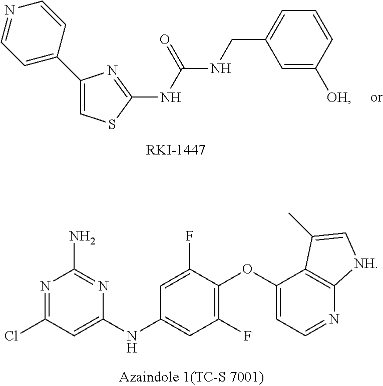 [SMILES: O=C(NCc1cccc(O)c1)Nc1nc(-c2ccncc2)cs1 ; Cc1c[nH]c2nccc(Oc3c(F)cc(Nc4cc(Cl)nc(N)n4)cc3F)c12]

Clause 3. The method of clause 1 or 2, wherein the Rho-kinase inhibitor is fasudil.
Clause 4. The method of any one of clauses 1-3, wherein the subject is a human.
Clause 5. The method of any one of clauses 1-4, wherein the subject is an animal.
Clause 6. The method of any one of clauses 1-5, wherein, before administering the therapeutically effective amount of the Rho-kinase inhibitor, the anti-fibrosis compound, or the pharmaceutically acceptable salt, ester, or solvate thereof, to the subject, a set of instructions to administer the therapeutically effective amount of the Rho-kinase inhibitor, the anti-fibrosis compound, or the pharmaceutically acceptable salt, ester, or solvate thereof, to the subject is generated by a computer-implemented method.
Clause 7. The method of any one of clauses 1-5, wherein the computer-implemented method comprises:
   executing, by one or more processors, a feedforward artificial neural network, wherein executing the feedforward artificial neural network comprises:
      receiving, by one or more processors, a plurality of patient parameters,
      the plurality of patient parameters comprising:
         one or more patients having diastolic dysfunction and one or more patients not having diastolic dysfunction,
         one or more patients having cardiac fibrosis and one or more patients not having cardiac fibrosis,
         one or more patients having pulmonary hypertension and one or more patients not having pulmonary hypertension,
         one or more patients having a left ventricular ejection fraction of ≥50%, and one or more patients not having a left ventricular ejection fraction of ≥50%,
         and
         one or more patients having cardiac amyloid deposits and one or more patients not having cardiac amyloid deposits,
      analyzing, by one or more processors, the plurality of patient parameters,
      arranging, by one or more processors, the plurality of patient parameters into neurons of an input layer of an artificial neural network,
      applying, by one or more processors, a weight to the plurality of patient parameters arranged in the input layer,
      arranging, by one or more processors, the weighted plurality of patient parameters to one or more hidden layers of the feedforward artificial neural network, and
      generating, by one or more processors, a plurality of optimized data sets of patient parameters;
   executing, by one or more processors, a cluster analysis, the executing comprising:
      receiving, by one or more processors, the plurality of optimized data sets of patient parameters,
   wherein the plurality of optimized data sets of patient parameters includes data of a plurality of patients;
      analyzing, by one or more processors, the plurality of optimized data sets of patient parameters,
      identifying, by one or more processors, a plurality of populations among the plurality of optimized data sets of patient parameters, and
      generating, by one or more processors, one or more clusters of the plurality of populations,
      analyzing, by one or more processors, the one or more clusters of the plurality of populations generated by the cluster analysis, and
      generating, by one or more processors, a set of instructions to administer the therapeutically effective amount of the Rho-kinase inhibitor, the anti-fibrosis compound, or the pharmaceutically acceptable salt, ester, or solvate thereof, to the subject.
Clause 8. The method of clause 7, wherein the plurality of patient parameters comprises data and metadata.
Clause 9. The method of clause 7 or 8, wherein the plurality of patient parameters further comprises age, weight, sex, height, and blood glucose levels.
Clause 10. A computer-implemented method, the computer implemented method comprising:
   executing, by one or more processors, a feedforward artificial intelligence, the executing the machine learning algorithm comprising:
      receiving, by one or more processors, a plurality of patient parameters, the plurality of patient parameters comprising:
         one or more patients having diastolic dysfunction and one or more patients not having diastolic dysfunction,
         one or more patients having cardiac fibrosis and one or more patients not having cardiac fibrosis,
         one or more patients having pulmonary hypertension and one or more patients not having pulmonary hypertension,
         one or more patients having a left ventricular ejection fraction of ≥50%, and one or more patients not having a left ventricular ejection fraction of ≥50%,
         and
         one or more patients having cardiac amyloid deposits and one or more patients not having cardiac amyloid deposits,
      analyzing, by one or more processors, the plurality of patient parameters,
      arranging, by one or more processors, the plurality of patient parameters into neurons of an input layer of an artificial neural network,
      applying, by one or more processors, a weight to the plurality of patient parameters arranged in the input layer,
      arranging, by one or more processors, the weighted plurality of patient parameters to one or more hidden layers of the feedforward artificial neural network, and
      generating, by one or more processors, a plurality of optimized data sets of patient parameters;
   executing, by one or more processors, a cluster analysis, the executing comprising:
      receiving, by one or more processors, a plurality of optimized data sets of patient parameters,
   wherein the plurality of optimized data sets of patient parameters includes data of a plurality of patients;
      analyzing, by one or more processors, the plurality of optimized data sets of patient parameters,
      identifying, by one or more processors, a plurality of populations among the plurality of optimized data sets of patient parameters, and
      generating, by one or more processors, one or more clusters of the plurality of populations,
      analyzing, by one or more processors, the one or more clusters of the plurality of populations generated by the cluster analysis, and
      generating, by one or more processors, a set of instructions to administer an effective amount of a Rho-kinase inhibitor, an anti-fibrosis compound, or a pharmaceutically acceptable salt, ester, or solvate thereof, to a subject experiencing:
         diastolic dysfunction,
         cardiac fibrosis,
         pulmonary hypertension, and
         a left ventricular ejection fraction of ≥50%,
   wherein the subject does not have cardiac amyloid deposits, and
      administering to the subject a therapeutically effective amount of the Rho-kinase inhibitor, the anti-fibrosis compound, or the pharmaceutically acceptable salt, ester or solvate thereof.
Clause 11. The computer-implemented method of clause 10, wherein the Rho-kinase inhibitor is:

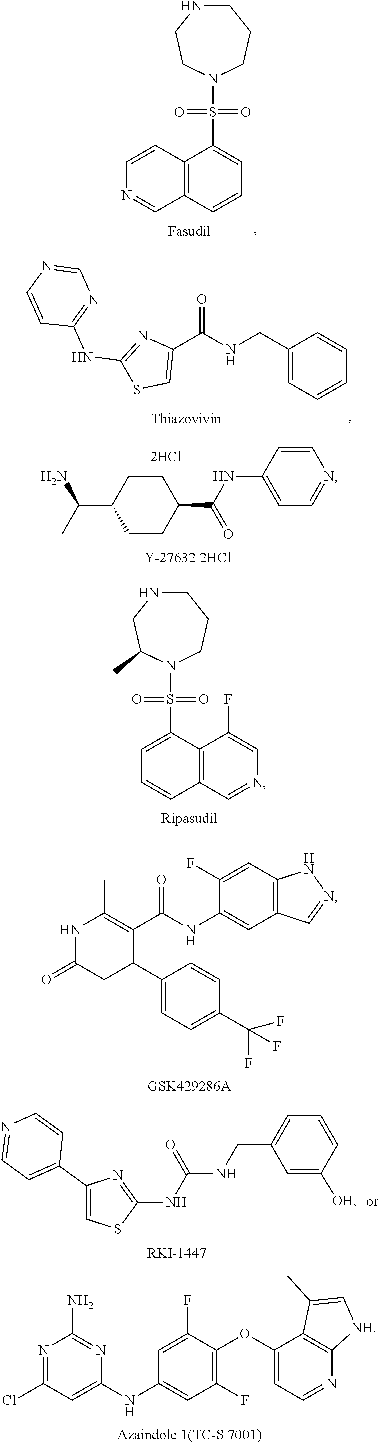

Clause 12. The computer-implemented method of clause 10 or 11, wherein the Rho-kinase inhibitor is fasudil.
Clause 13. The computer-implemented method of any one of clauses 10-12, wherein the subject is a human.
Clause 14. The computer-implemented method of any one of clauses 10-12, wherein the subject is an animal.
Clause 15. The computer-implemented method of any one of clauses 10-14, wherein the plurality of patient parameters comprises data and metadata.
Clause 16. The computer-implemented method of any one of clauses 10-15, wherein the plurality of patient parameters further comprises age, weight, sex, height, and blood glucose levels.
Clause 17. A method of reducing the stiffness of heart tissue, the method comprising:
   administering a therapeutically effective amount of a Rho-kinase inhibitor, an anti-fibrosis compound, or a pharmaceutically acceptable salt, ester, or solvate thereof, to a subject experiencing:
      diastolic dysfunction,
      cardiac fibrosis,
      pulmonary hypertension, and
      a left ventricular ejection fraction of ≥50%,
wherein the subject does not have cardiac amyloid deposits.
Clause 18. The method of clause 17, wherein the Rho-kinase inhibitor is:

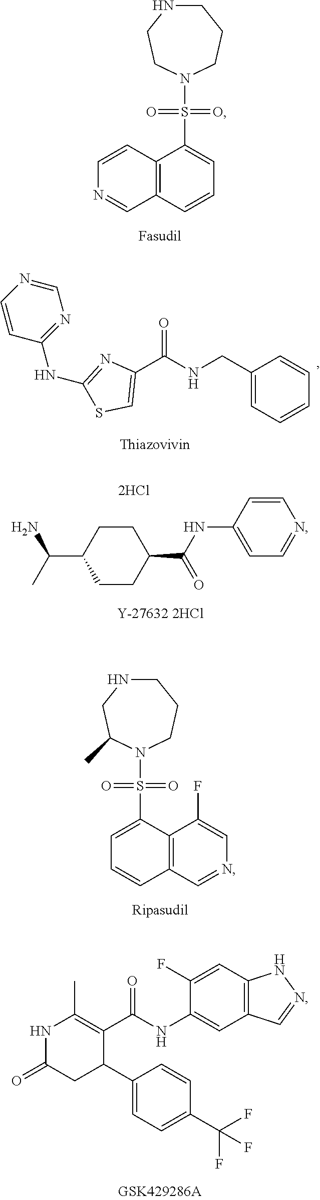 [SMILES: O=S(=O)(c1cccc2cnccc12)N1CCCNCC1 ; O=C(NCc1ccccc1)c1csc(Nc2ccncn2)n1 ; C[C@@H](N)[C@H]1CC[C@H](C(=O)Nc2ccncc2)CC1 ; C[C@H]1CNCCCN1S(=O)(=O)c1cccc2cncc(F)c12 ; CC1=C(C(=O)Nc2cc3cn[nH]c3cc2F)C(c2ccc(C(F)(F)F)cc2)CC(=O)N1]

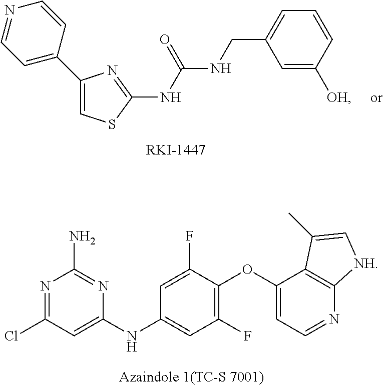 [SMILES: O=C(NCc1cccc(O)c1)Nc1nc(-c2ccncc2)cs1 ; Cc1c[nH]c2nccc(Oc3c(F)cc(Nc4cc(Cl)nc(N)n4)cc3F)c12]

Clause 19. The method of clause 17 or 18, wherein the Rho-kinase inhibitor is fasudil.
Clause 20. The method of any one of clauses 17-19, wherein, before administering the therapeutically effective amount of the Rho-kinase inhibitor, the anti-fibrosis compound, or the pharmaceutically acceptable salt, ester, or solvate thereof, to the subject, a set of instructions to administer the therapeutically effective amount of the Rho-kinase inhibitor, the anti-fibrosis compound, or the pharmaceutically acceptable salt, ester, or solvate thereof, to the subject is generated by a computer-implemented method.

What is claimed is:

1. A method of treating heart failure with preserved ejection fraction (HFpEF), the method comprising:
administering a therapeutically effective amount of a Rho-kinase inhibitor, an anti-fibrosis compound, or a pharmaceutically acceptable salt, ester, or solvate thereof, to a subject experiencing:
diastolic dysfunction,
cardiac fibrosis,
pulmonary hypertension, and
a left ventricular ejection fraction of ≥50%,
wherein the subject does not have cardiac amyloid deposits.

2. The method of claim 1, wherein the Rho-kinase inhibitor is:

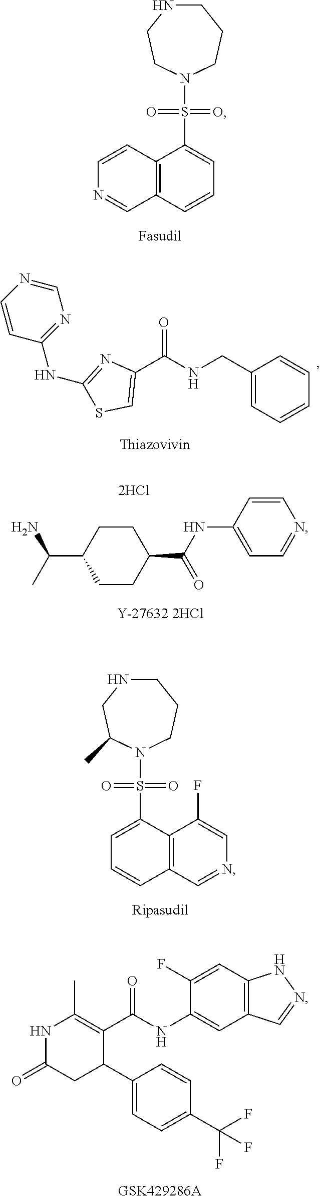 [SMILES: O=S(=O)(c1cccc2cnccc12)N1CCCNCC1 ; O=C(NCc1ccccc1)c1csc(Nc2ccncn2)n1 ; C[C@@H](N)[C@H]1CC[C@H](C(=O)Nc2ccncc2)CC1 ; C[C@H]1CNCCCN1S(=O)(=O)c1cccc2cncc(F)c12 ; CC1=C(C(=O)Nc2cc3cn[nH]c3cc2F)C(c2ccc(C(F)(F)F)cc2)CC(=O)N1]

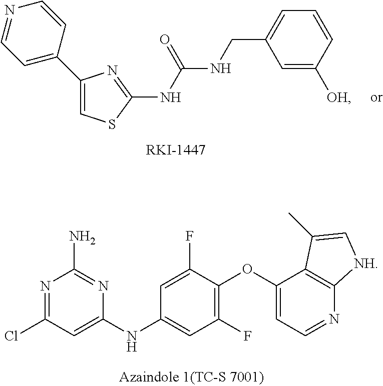 [SMILES: O=C(NCc1cccc(O)c1)Nc1nc(-c2ccncc2)cs1 ; Cc1c[nH]c2nccc(Oc3c(F)cc(Nc4cc(Cl)nc(N)n4)cc3F)c12]

3. The method of claim 1, wherein the Rho-kinase inhibitor is fasudil.

4. The method of claim 1, wherein the subject is a human.

5. The method of claim 1, wherein the subject is an animal.

6. The method of claim 1, wherein, before administering the therapeutically effective amount of the Rho-kinase inhibitor, the anti-fibrosis compound, or the pharmaceutically acceptable salt, ester, or solvate thereof, to the subject, a set of instructions to administer the therapeutically effective amount of the Rho-kinase inhibitor, the anti-fibrosis compound, or the pharmaceutically acceptable salt, ester, or solvate thereof, to the subject is generated by a computer-implemented method.

7. The method of claim 6, wherein the computer-implemented method comprises:
executing, by one or more processors, a feedforward artificial neural network, wherein executing the feedforward artificial neural network comprises:
receiving, by one or more processors, a plurality of patient parameters,
the plurality of patient parameters comprising:
one or more patients having diastolic dysfunction and one or more patients not having diastolic dysfunction,
one or more patients having cardiac fibrosis and one or more patients not having cardiac fibrosis,
one or more patients having pulmonary hypertension and one or more patients not having pulmonary hypertension,
one or more patients having a left ventricular ejection fraction of ≥50%, and one or more patients not having a left ventricular ejection fraction of ≥50%,
and
one or more patients having cardiac amyloid deposits and one or more patients not having cardiac amyloid deposits,
analyzing, by one or more processors, the plurality of patient parameters,
arranging, by one or more processors, the plurality of patient parameters into neurons of an input layer of an artificial neural network,
applying, by one or more processors, a weight to the plurality of patient parameters arranged in the input layer,
arranging, by one or more processors, the weighted plurality of patient parameters to one or more hidden layers of the feedforward artificial neural network, and
generating, by one or more processors, a plurality of optimized data sets of patient parameters;
executing, by one or more processors, a cluster analysis, the executing comprising:
receiving, by one or more processors, a plurality of optimized data sets of patient parameters,
wherein the plurality of optimized data sets of patient parameters includes data of a plurality of patients;
analyzing, by one or more processors, the plurality of optimized data sets of patient parameters,
identifying, by one or more processors, a plurality of populations among the plurality of optimized data sets of patient parameters, and
generating, by one or more processors, one or more clusters of the plurality of populations,
analyzing, by one or more processors, the one or more clusters of the plurality of populations generated by the cluster analysis, and
generating, by one or more processors, a set of instructions to administer the therapeutically effective amount of the Rho-kinase inhibitor, the anti-fibrosis compound, or the pharmaceutically acceptable salt, ester, or solvate thereof, to the subject.

8. The method of claim 7, wherein the plurality of patient parameters comprises data and metadata.

9. The method of claim 7, wherein the plurality of patient parameters further comprises age, weight, sex, height, and blood glucose levels.

10. A computer-implemented method, the computer implemented method comprising:
executing, by one or more processors, a feedforward artificial intelligence, the executing the machine learning algorithm comprising:
receiving, by one or more processors, a plurality of patient parameters, the plurality of patient parameters comprising:
one or more patients having diastolic dysfunction and one or more patients not having diastolic dysfunction,
one or more patients having cardiac fibrosis and one or more patients not having cardiac fibrosis,
one or more patients having pulmonary hypertension and one or more patients not having pulmonary hypertension,
one or more patients having a left ventricular ejection fraction of ≥50%, and one or more patients not having a left ventricular ejection fraction of ≥50%,
and
one or more patients having cardiac amyloid deposits and one or more patients not having cardiac amyloid deposits,
analyzing, by one or more processors, the plurality of patient parameters,
arranging, by one or more processors, the plurality of patient parameters into neurons of an input layer of an artificial neural network,
applying, by one or more processors, a weight to the plurality of patient parameters arranged in the input layer,
arranging, by one or more processors, the weighted plurality of patient parameters to one or more hidden layers of the feedforward artificial neural network, and
generating, by one or more processors, a plurality of optimized data sets of patient parameters;
executing, by one or more processors, a cluster analysis, the executing comprising:
receiving, by one or more processors, a plurality of optimized data sets of patient parameters,
wherein the plurality of optimized data sets of patient parameters includes data of a plurality of patients;
analyzing, by one or more processors, the plurality of optimized data sets of patient parameters,
identifying, by one or more processors, a plurality of populations among the plurality of optimized data sets of patient parameters, and
generating, by one or more processors, one or more clusters of the plurality of populations,
analyzing, by one or more processors, the one or more clusters of the plurality of populations generated by the cluster analysis, and
generating, by one or more processors, a set of instructions to administer an effective amount of a Rho-kinase inhibitor, an anti-fibrosis compound, or a pharmaceutically acceptable salt, ester, or solvate thereof, to a subject experiencing:
diastolic dysfunction,
cardiac fibrosis,
pulmonary hypertension, and
a left ventricular ejection fraction of ≥50%,
wherein the subject does not have cardiac amyloid deposits, and
administering to the subject a therapeutically effective amount of the Rho-kinase inhibitor, the anti-fibrosis compound, or the pharmaceutically acceptable salt, ester or solvate thereof.

11. The computer-implemented method of claim 10, wherein the Rho-kinase inhibitor is:

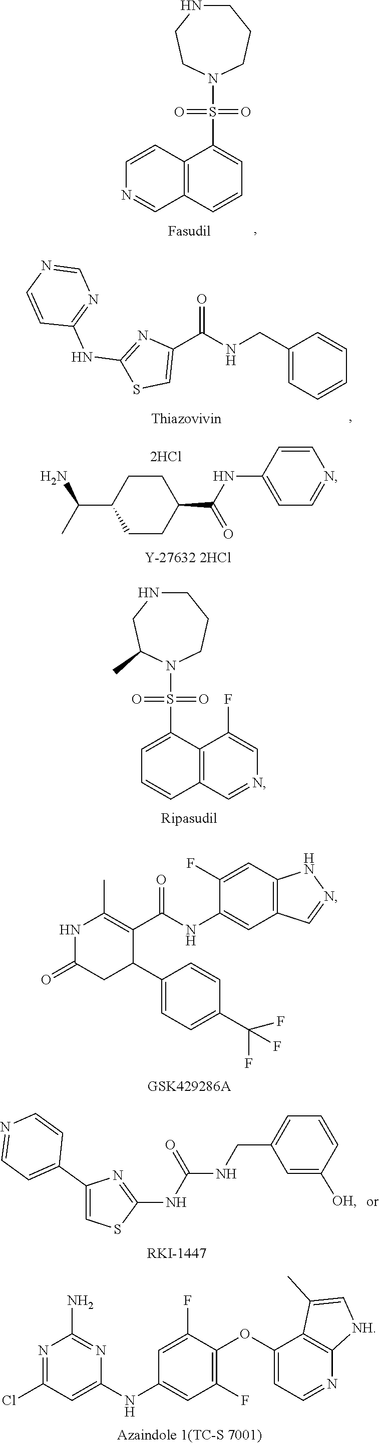

12. The computer-implemented method of claim 10, wherein the Rho-kinase inhibitor is fasudil.

13. The computer-implemented method of claim 10, wherein the subject is a human.

14. The computer-implemented method of claim 10, wherein the subject is an animal.

15. The computer-implemented method of claim 10, wherein the plurality of patient parameters comprises data and metadata.

16. The computer-implemented method of claim 10, wherein the plurality of patient parameters further comprises age, weight, sex, height, and blood glucose levels.

17. A method of reducing the stiffness of heart tissue, the method comprising:
administering a therapeutically effective amount of a Rho-kinase inhibitor, an anti-fibrosis compound, or a pharmaceutically acceptable salt, ester, or solvate thereof, to a subject experiencing:
diastolic dysfunction,
cardiac fibrosis,
pulmonary hypertension, and
a left ventricular ejection fraction of ≥50%,
wherein the subject does not have cardiac amyloid deposits.

18. The method of claim 17, wherein the Rho-kinase inhibitor is:

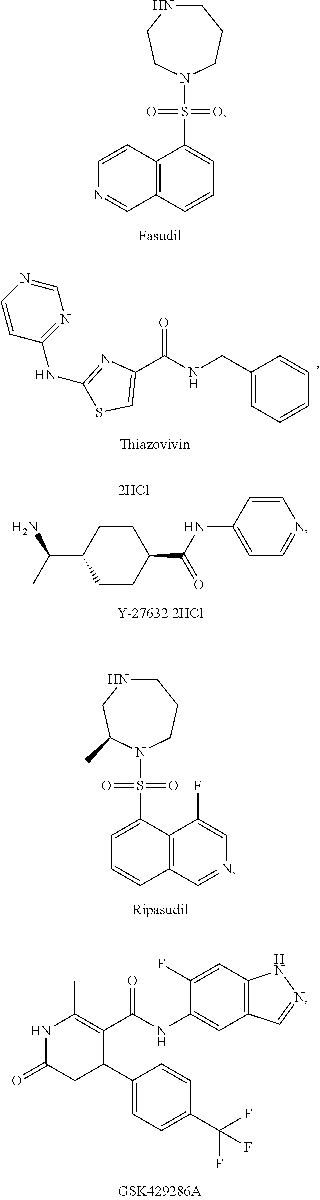 [SMILES: O=S(=O)(c1cccc2cnccc12)N1CCCNCC1 ; O=C(NCc1ccccc1)c1csc(Nc2ccncn2)n1 ; C[C@@H](N)[C@H]1CC[C@H](C(=O)Nc2ccncc2)CC1 ; C[C@H]1CNCCCN1S(=O)(=O)c1cccc2cncc(F)c12 ; CC1=C(C(=O)Nc2cc3cn[nH]c3cc2F)C(c2ccc(C(F)(F)F)cc2)CC(=O)N1]

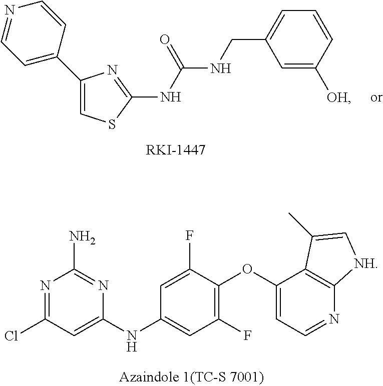 [SMILES: O=C(NCc1cccc(O)c1)Nc1nc(-c2ccncc2)cs1 ; Cc1c[nH]c2nccc(Oc3c(F)cc(Nc4cc(Cl)nc(N)n4)cc3F)c12]

19. The method of claim 17, wherein the Rho-kinase inhibitor is fasudil.

20. The method of claim 17, wherein, before administering the therapeutically effective amount of the Rho-kinase inhibitor, the anti-fibrosis compound, or the pharmaceutically acceptable salt, ester, or solvate thereof, to the subject, a set of instructions to administer the therapeutically effective amount of the Rho-kinase inhibitor, the anti-fibrosis compound, or the pharmaceutically acceptable salt, ester, or solvate thereof, to the subject is generated by a computer-implemented method.
